SEQUENCE LISTING (86 sequences)

SEQ ID NO: 1 - protein, 26 aa, Mus musculus
MENVSNPGSCIEEGEERGRILGSPFL

SEQ ID NO: 2 - protein, 19 aa, Mus musculus
MGEYLAEPRGFVCGVEPLC

SEQ ID NO: 3 - protein, 4 aa, Mus musculus
MAWH

SEQ ID NO: 4 - protein, 19 aa, Mus musculus
MEAGWEVQHWVSDMECLTV

SEQ ID NO: 5 - protein, 6 aa, Mus musculus
MECLTV

SEQ ID NO: 6 - protein, 3 aa, Mus musculus
MTV

SEQ ID NO: 7 - protein, 13 aa, Mus musculus
MCGSEEVFVVESA

SEQ ID NO: 8 - protein, 92 aa, Mus musculus
MACYQMYFTGRKVDEPSELGSGLELSYFHTGGSSQAVGLFIENMISTSHGHFQEMQFSIWSFTVLQISAPGSHLVPETERAEGPGVFVEHDI

SEQ ID NO: 9 - protein, 87 aa, Mus musculus
MYFTGRKVDEPSELGSGLELSYFHTGGSSQAVGLFIENMISTSHGHFQEMQFSIWSFTVLQISAPGSHLVPETERAEGPGVFVEHDI

SEQ ID NO: 10 - protein, 49 aa, Mus musculus
MISTSHGHFQEMQFSIWSFTVLQISAPGSHLVPETERAEGPGVFVEHDI

SEQ ID NO: 11 - protein, 38 aa, Mus musculus
MQFSIWSFTVLQISAPGSHLVPETERAEGPGVFVEHDI

SEQ ID NO: 12 - protein, 21 aa, Mus musculus
MWWGLILSASVKFLQRKEILC

SEQ ID NO: 13 - protein, 14 aa, Mus musculus
MVGADLCKGGWHCV

SEQ ID NO: 14 - protein, 13 aa, Mus musculus
MREPVKNLQGLVS

SEQ ID NO: 15 - protein, 25 aa, Mus musculus
MEVYELRVTLMETGRERSHFVKTSL

SEQ ID NO: 16 - protein, 15 aa, Mus musculus
METGRERSHFVKTSL

SEQ ID NO: 17 - protein, 30 aa, Homo sapiens
MGLSAVGRTRAESGTAERAAPVFVLGLQAV

SEQ ID NO: 18 - protein, 24 aa, Homo sapiens
MLLWDPSGFQQISIKKVISKTLPT

SEQ ID NO: 19 - protein, 26 aa, Homo sapiens
MLPNTMGQLVEGGHMKQVLSKAVLTV

SEQ ID NO: 20 - protein, 21 aa, Homo sapiens
MGQLVEGGHMKQVLSKAVLTV

SEQ ID NO: 21 - protein, 12 aa, Homo sapiens
MKQVLSKAVLTV

SEQ ID NO: 22 - protein, 4 aa, Homo sapiens
MSEC

SEQ ID NO: 23 - protein, 11 aa, Homo sapiens
MAHFVAVQITV

SEQ ID NO: 24 - protein, 6 aa, Homo sapiens
MGICYS

SEQ ID NO: 25 - protein, 18 aa, Homo sapiens
MKRAGEGKSFCKGRHYSV

SEQ ID NO: 26 - protein, 21 aa, Homo sapiens
MLTTLIYYQGNSVIFVRQHSA

SEQ ID NO: 27 - protein, 37 aa, Homo sapiens
MQLPHFVARLFPHEQFVFIQQLSSLGKPFCRGVCHSV

SEQ ID NO: 28 - protein, 11 aa, Homo sapiens
MGFSKGRKCCG

SEQ ID NO: 29 - protein, 18 aa, Homo sapiens
MKKIWLSRKVFLYWAETL

SEQ ID NO: 30 - protein, 34 aa, Homo sapiens
MGKVHVMPLLFMESKAASINGNIMLVYVETHNTV

SEQ ID NO: 31 - protein, 28 aa, Homo sapiens
MPLLFMESKAASINGNIMLVYVETHNTV

SEQ ID NO: 32 - protein, 23 aa, Homo sapiens
MESKAASINGNIMLVYVETHNTV

SEQ ID NO: 33 - protein, 11 aa, Homo sapiens
MLVYVETHNTV

SEQ ID NO: 34 - protein, 55 aa, Homo sapiens
MEEGSFIYTIKGPWMTHSLCDCCVIGFQTLALIGIIGEGTWWLLQGVFCLGRTHC

SEQ ID NO: 35 - protein, 41 aa, Homo sapiens
MTHSLCDCCVIGFQTLALIGIIGEGTWWLLQGVFCLGRTHC

SEQ ID NO: 36 - protein, 16 aa, Homo sapiens
MESQATGFCYEASHSV

SEQ ID NO: 37 - protein, 12 aa, Mus musculus
LAEPRGFVCGVE

SEQ ID NO: 38 - DNA, 773 bp, Mus musculus
  Intron (9)..(108): intron 5 prime to J beta 2.6
ttccctaaatgggagaatacctcgctgaaccccgcgggtttgtgtgtggggttgagcctctgtgctcctatgaacagtacttcggtcccggcaccaggctcacggttttagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctgagacaaacttttatgcatcctgagccgttcttcaccctggccatagattttcctgcaccttctctaattcctgttcctaagaacttgtctcttcttcctccatggatatccatccttcctcgttgacaccttgactctgaaa

SEQ ID NO: 39 - protein, 207 aa, Mus musculus
MGEYLAEPRGFVCGVEPLCSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 40 - protein, 129 aa, Mus musculus
  misc_feature (1)..(129): J beta 2.1 sequence
KGSREVEPPFSPYHVNHQQSIRTCMGNYELIKKHVEKTLCGKEVTSPFSLEATWTPTGSLQISNSLCQTLSEMDIRSQAKSGISSSIDRPHARSRLPYQFWRMENVSNPGSCIEEGEERGRILGSPFLL

SEQ ID NO: 41 - protein, 54 aa, Mus musculus
  misc_feature (1)..(54): J beta 2.6 sequence
ELLGNCSGEFWGFWRLYPEFPSRALEREAEQGDFPMGEYLAEPRGFVCGVEPLC

SEQ ID NO: 42 - protein, 340 aa, Mus musculus
  misc_feature (1)..(340): J alpha TA31 sequence
VSKKKKKKKSVTILNSEPAEGAINSSLLGSLDPGNVLEHCTGLLPSPKDDPCQDRSSFLWGGGQWIFAVIVFCLAHSPRLWPETSPQSTTQEQRVKGLNGERDIGHVRTRRNFTQKKNCHLGRCSVSMAEVTPPPCPRLVSQLRHGHQKGGFLSSLKTNLAESHLPSSPNEPVVSVDALGSVRRVFAVAEGSRLTRRARWGRTYRGWTEASPCLHSSCAASSCGFTGGRGGWGRGAIPKAVACFGICSGLLCLPPWERTHLASRRLDVAGQEDTGVGGNSFRGEGERGGRTVVEGVTGGSMSRMSEVKFKKLEIKNKKQGRGLQKVYRAGTVDFVMAWHT

SEQ ID NO: 43 - protein, 253 aa, Mus musculus
  misc_feature (1)..(253): J alpha TA46 sequence
VFLPGRWEPKEVDRDISNPPCKPLVLPTVDTVTIRTLSHIDEGSDVVHTEDSRDLSLVTVSDCMPIVVHSRVQQTKDRDIKIRWTLSPHLCNQMIFTGSLANGCVASLTISPLLSPWLSFGSLSLTNLKSIYIIRFLGCITHKKMTSRHININPEERGQRALSQTCSELNLTTPCFNQLASAYDQLRQRATDRKWSSRHHLTRALPHQRYFRVQESFPQAGWLERGHGSALRQAMEAGWEVQHWVSDMECLTV

SEQ ID NO: 44 - protein, 310 aa, Mus musculus
  misc_feature (1)..(310): J alpha New05 sequence
VKDGYPKTKVCGFAVLCSFGGCMSLPPRSLCITLMGLCLMKSGHSKDLDEEVIIITAFFHYLRIRSARFINVRLMFVLRYKPNNSKIRLSSVTTHIHTHSHTHILTHWHNHTHTHTLSQSHTHTHSHTSTITHTLTQPHTHSLSLSLSLSLSLSLSLSLSLPRQCNCIWFPSRNGCCVCLTDMQSYQLVSWLGFCYCFSVKTLPVKEAWCYQPSCHYSNHIYTPFYYFISLKLAQLIRIQCWGNKTSGFSSSELHSQLLVLRGCSKPSQTLGTKAARRKASTRGEDDVAFLGLPLGPSCLLVIVRPQMTV

SEQ ID NO: 45 - protein, 688 aa, Mus musculus
  misc_feature (1)..(688): J alpha S 58 sequence
WVRFHVTAVALCSFTSLLHLFLETLGFRLSFLFKKQSLSKQDLLCLLSFHIVTKAGRICSKLGLRLLAKVEWMVLVYRKERFVLLFFPYSKVKATTVASKVLQAWSVLQGETWGNWLTFHGKTGMLFVVGLLLLLLSSLSLSLKETYNTFLSGFELGIQMCITCSWQGSRAVVLNLPNVVAPSPPKTIKLFCCYFIAVTLLLLIGMISYMQLIYATPVKGSLNPQRRSALQDESRCCRGRWSTVSNVRGAIELGRNTMPTFEEKKNSSLGLEQDPLFLVSPLPLEKKPFICNGLSRLMSFMRFHVLTDSLGRRSLLPLQVVFDVGNVNCTAKIRRAGINSQPLLMLSLNRNQIRMLSSVCVHTPPRASFDCQLIQIFRHLSEQTSLGSLCLNLSRYLHNCQICFTLCCIDSAKQMRLCFPRSFSPRRSSLPPSKHLFTQREDVQRVTLIAAASLHLYDSLPWKRLKHFIRLISTDQPNEERNRFASFLWLQFQATHLEHLVRHLRNTGARREVVSLCGLVFLSCTENFTQEEESKVENQPGIHMYTKQSASALSGSTVWFPHSPTPAPFISNTYIILFSFSFEFLSAMPSHNPSTYHCLSNPRMDGSGTGRVLFSGPSAEPLKKCRLYPSSVATRRLGRGQDEEKPQESGTASLWYIRLNLLSGLKCFSFHLEPMCGSEEVFVVESAT

SEQ ID NO: 46 - protein, 275 aa, Mus musculus
  misc_feature (1)..(275): J alpha New06 sequence
KCVFSCSLGLEQYCSLHPQIFSRRIQCLALQTLPVPLKGSYSFFKHRRIPFNVANCGGDTAQGPNLCSSLLGQLCLLSHRTSESGGLFPSLAFPVDEVVLSTNFIVKDTHDRQLLPYFSLNKFFLCLQHISANEFLVIQINSSVTTVASYPIIQNSLTHHSAAAHCASSNPDLHASSNKAKRMACYQMYFTGRKVDEPSELGSGLELSYFHTGGSSQAVGLFIENMISTSHGHFQEMQFSIWSFTVLQISAPGSHLVPETERAEGPGVFVEHDIT

SEQ ID NO: 47 - protein, 556 aa, Mus musculus
  misc_feature (1)..(556): J alpha New08 sequence
VMFHFLMFNSLPLSRCSECRVGKLHMLGHGGQHSCTGYSTAQPDTTSPTTGETAPTLPPDTKIFLIVYLIRAKGKIKKLCPESILKSPRPSPPYPHSPADCKFNVIFGSYKGFLCLMTPTVSLPSFIKGLLFCVWPLLASWFCPHAPLCLFQGWAGDNSFKSHFDVTDNRDKVLAKCNTAHGVFSRHTTSQLFSSVQKHGHSYLMSAIYSDTAKCSFKAGTRDFLWDLFLRLTMGWAFSGSSEMPSWIPALPMEILWSGTAKPDMFLLYRLLQGLEIRTLRENKSFGMGRLLDGSIRKRNDQEERPKKNTGQALGWGGVGMSRKMVTVGIQEAGSLSEGKQGFLLKVPSQLSNLNQQGHLPFPSDFPVHVGMPLPPTMVCEVGRGIDQEYVHSGPLFKHETPESVRGAKSLGPRREMQQSNSSQQVWRSTEQDPVLALCLTPLASPDHTAHPSSFSPQESKVLDREPEIPPGQVQKGWSGAQGWFLKTLWISIFLIYNKFLSVIRKMFLLTIPVKGKDNIYRGPLLRCQFPPWASMWWGLILSASVKFLQRKEILC

SEQ ID NO: 48 - protein, 604 aa, Mus musculus
  misc_feature (1)..(604): J alpha LB2A sequence
VIVTHPLCIPPTRSIFALSSLLGSLSNVVSVTPCPYLLSRYKWSKQILGFHHSETDNCVLDILQKEGFQSKGSHYFYLTHKEAGDNWKVPGEYLGFQKADMAQCMHSKIPLTFIEYLLYACVNAPCTLSHLRGWLWGRFYPTFKGKVEIVTKWLRENGGPSTSSRPGCPHCGLSQPGSCGLYRMKPVVLVTTSSVLSQPCLEQGVRDSLCFLDSDTLKQNGECVHEQFHSGSMVNGQTNLKRSSLWLESPFSTPLSSLPTFLSSWTFISGKPLHRCLCRSQIKNERLSPGHTKNLRRLFFQYLKNSCVDNGRGHQRQNQKQMKRRPSFSGMLLNGAVGGQAPLSLESALQGLHSGSSGLRWRALWKEFLWHFRLWISCELEVLRPHDPSIEDKRVGYICFFLFLLFPRNRPSNCSQAEAYRDFFTLRRRTMISQCSKWGKKRREREREREREREREREREREREMPRRARGTKEVGLCRGQISIEVFISSALENPSIMVLVTEAVFTGKQDQGSEGLPITLSKGCVIAFERTLAVERLLLPQIICLLRCSLRKSDCLPLLGAWGKDLGKLRADRRSFSALHSQARERGWGMVGADLCKGGWHCV

SEQ ID NO: 49 - protein, 385 aa, Mus musculus
  misc_feature (1)..(385): J alpha DK1 sequence
VCLFLWIPNLIHCDKCKLFRHVSGVSTVPIHPDITGSKVPSHAFPVLTRKTGSSLYCWQAQGSRLEDASDAQQPAWDCPGRESCSEMPSSLPLGIILLSSPTARPCLSVAYSIPASHTCGCANILIEASGRSGSSMLLFGKASHSKAGLDSPPPKSLHIPGSGLQVQTTMLVFVVLDMEPGCACLQGKHFIGAISLAHLPVSIFFERISWYSHLVHRQKDDVDVPRWHTVIWSQALIFPPSIFRCLSVKVISSSMSPGGRLACCPSSAVAWMASSCYPTLCIPIIHLTLYVYLLFPYSMYCHATVMLFIVSSVSSVVPITKIQRPNCLPCLKIIVLEKKLEFCCCLYRHELRSLAVARTGYDFCSVLHTPVMREPVKNLQGLVSL

SEQ ID NO: 50 - protein, 399 aa, Mus musculus
  misc_feature (1)..(399): J alpha TA39 sequence
VPDSWLRPPLSHSLYHTDDHMPYHSSKVELGFNEERNMLLVVAVLHPMSHSMFIITLITSSDKRKFTRRTVTICTLVKMKVSTGAGAYCNSGYQKDQALARKKLNKVDLVKLLQIFFKNQYVSELTGEYSAAILSGFSYSYGTTVVEPCKRGFHGLNSMLSLYSSNQKGGIPSRTPKREESMLITSIDHSRLSIFVRQHGTTIYNVFIWGTRHHRDAGCDPLNLPQYLGTVVKELMVHADKHIPCMGKLSKGCRTGCEQDRSCRNPRNNSSRRADPEERAAQLKHIQVPICFDSCTGPALSVKRKCLIILHKLIGVNVCKNILQILKCYPHIKYGSIKQQKILKLGQSTLLRRDGVCSCGSVATGTGKHPLSLMEVYELRVTLMETGRERSHFVKTSLT

SEQ ID NO: 51 - protein, 225 aa, Homo sapiens [sequence omitted]

SEQ ID NO: 52 - protein, 39 aa, Homo sapiens [sequence omitted]

SEQ ID NO: 53 - protein, 142 aa, Homo sapiens [sequence omitted]

SEQ ID NO: 54 - protein, 162 aa, Homo sapiens
  misc_feature (1)..(162): J alpha 6 (79270 to 79331), containing [SEQ ID NO19], [SEQ ID NO20], [SEQ ID NO 21]
QLQEKRHIKFPLLSVLAALSEAPCILKSSRARPSECLPQASRVWCLYWGAGSRHGELLPCFSADGKVVFSPGYTGAKELSSPQPLAPAPGLQHSGALRTAVGDFLQLREYSGGFPRMLPNTMGQLVEGGHMKQVLSKAVLTVCIRRKLHTYIWKRNQPYCSS

SEQ ID NO: 55 - protein, 133 aa, Homo sapiens [sequence omitted]

SEQ ID NO: 56 - protein, 158 aa, Homo sapiens [sequence omitted]

SEQ ID NO: 57 - protein, 168 aa, Homo sapiens [sequence omitted]

SEQ ID NO: 58 - protein, 170 aa, Homo sapiens [sequence omitted]

SEQ ID NO: 59 - protein, 163 aa, Homo sapiens [sequence omitted]

SEQ ID NO: 60 - protein, 142 aa, Homo sapiens [sequence omitted]

SEQ ID NO: 61 - protein, 176 aa, Homo sapiens
  misc_feature (1)..(176): J alpha 25 (59046 to 59105)
QKDKASPLSLGRGQGCLSSQAQAGGRKLGVFAEPRNTVGITMVRILSLVPEPDCPCCPVSTVKWRKMSPVLDVGRSCRVLRPGVHRDLRSGDGEEGKRNEKQNHKDNTEEGFIFGKENHKAVLTLEEMHSFGGSLLRRALCRGKLSCVFDAEIITMQKDKASPLSLGRGQGCLSSQ

SEQ ID NO: 62 - protein, 141 aa, Homo sapiens [sequence omitted]

SEQ ID NO: 63 - protein, 148 aa, Homo sapiens [sequence omitted]

SEQ ID NO: 64 - protein, 144 aa, Homo sapiens [sequence omitted]

SEQ ID NO: 65 - protein, 152 aa, Homo sapiens [sequence omitted]

SEQ ID NO: 66 - protein, 141 aa, Homo sapiens [sequence omitted]

SEQ ID NO: 67 - DNA, 678 bp, Homo sapiens
  Intron (1)..(90): intron 5 prime to J beta 2.3
atggggctctcagcggtgggaaggacccgagctgagtctgggacagcagagcgggcagcaccggtttttgtcctgggcctccaggctgtgagcacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 68 - protein, 225 aa, Homo sapiens [sequence omitted]

SEQ ID NO: 69 - DNA, 20 bp, Mus musculus
  misc_feature (1)..(20): exonic J beta 2.6 primer
ctatgaacagtacttcggtc

SEQ ID NO: 70 - DNA, 19 bp, Mus musculus
  misc_feature (1)..(19): intronic J beta 2.6 primer
atgggagaatacctcgctg

SEQ ID NO: 71 - DNA, 19 bp, Mus musculus
ccctaaatgggagaatacc

SEQ ID NO: 72 - DNA, 30 bp, Mus musculus
  misc_feature (1)..(30): antisense primer C beate 3
catcctatcatcagggggttctgtctgcaa

SEQ ID NO: 73 - DNA, 29 bp, Homo sapiens
  misc_feature (1)..(29): sense primer
ccggaattccatggggctctcagcggtgg

SEQ ID NO: 74 - DNA, 31 bp, Homo sapiens
  misc_feature (1)..(31): antisense primer
cgcggatccctagcctctggaatcctttctc

SEQ ID NO: 75 - DNA, 19 bp, Mus musculus
  misc_feature (1)..(19): sense primer for CD3 epsilon
tgccctctagacagtgacg

SEQ ID NO: 76 - DNA, 19 bp, Mus musculus
  misc_feature (1)..(19): antisense primer for CD3 epsilon
cttccggttccggttcgga

SEQ ID NO: 77 - DNA, 30 bp, Mus musculus
atgtgactccacccaaggtctccttgtttg

SEQ ID NO: 78 - DNA, 30 bp, Mus musculus
aaggctaccctcgtgtgcttggccaggggc

SEQ ID NO: 79 - DNA, 30 bp, Mus musculus; identical to SEQ ID NO: 72

SEQ ID NO: 80 - DNA, 30 bp, Mus musculus; identical to SEQ ID NO: 72

SEQ ID NO: 81 - DNA, 28 bp, Mus musculus
ttcagagtcaaggtgtcaacgaggaagg

SEQ ID NO: 82 - DNA, 26 bp, Mus musculus
aagatcctcggtctcaggacagcacc

SEQ ID NO: 83 - DNA, 30 bp, Mus musculus
actgtgctggacatgaaagctatggattcc

SEQ ID NO: 84 - DNA, 20 bp, Mus musculus
gatttaacctgctcatgacg

SEQ ID NO: 85 - protein, 12 aa, Mus musculus
RGGGGGRGGLHD

SEQ ID NO: 86 - protein, 4 aa, Mus musculus
MMLV

(19) United States
(12) Patent Application Publication
(10) Pub. No.: US 2004/0259196 A1
(43) Pub. Date: Dec. 23, 2004

(54) T cell receptor variants expressed in mesenchymal cells and uses thereof

(76) Inventors: Dov Zipori, Rehovot (IL); Arie Leon Rozenszajn, Ramat Hasharon (IL); Mira Barda-Saad, Ganei Tiqvah (IL); Yaron Shav-Tal, Elkanah (IL)

Correspondence Address: WINSTON & STRAWN, PATENT DEPARTMENT, 1400 L STREET, N.W., WASHINGTON, DC, 20005-3502, US

(21) Appl. No.: 10/642,642

(22) Filed: Aug. 19, 2003

Related U.S. Application Data
(63) Continuation of application No. PCT/IL02/00130, filed on Feb. 20, 2002.

(30) Foreign Application Priority Data
Feb. 20, 2001 (IL) 141539

(51) Int. Cl. C07K 14/705; C07H 21/04

(52) U.S. Cl. 435/69.1; 435/320.1; 435/325; 530/350; 536/23.5

(57) ABSTRACT

The present invention concerns novel polynucleotide transcripts of T cell receptor (TCR) genes as well as amino acid sequences encoded by these transcripts, and their use in the modulation of mesenchymal cell growth. It further relates to the novel proteins, or peptides encoded by these transcripts, and uses thereof. The invention also concerns cDNA molecules encoded by a T cell receptor (TCR) gene, these novel cDNA molecules lacking V region sequences and comprising a constant (C) domain and joining (J) region sequences, and a 5′ intronic J sequence upstream to said J region sequence including an in-frame methionine codon.

### CROSS-REFERENCE TO RELATED APPLICATIONS

This application is a continuation of the International Application PCT/IL02/00130 filed Feb. 20, 2002, the entire content of which is expressly incorporated herein by reference thereto, which application claims priority to Israel Patent Application No. 141539 filed Feb. 20, 2001.

### FIELD OF THE INVENTION

The present invention relates to polynucleotide transcripts comprising intronic sequences of T cell receptor (TCR) genes expressed in mesenchymal cells, to antisense polynucleotides of these and uses thereof in the modulation of mesenchymal cell growth. It further relates to the novel proteins, or peptides encoded by these transcripts, and uses thereof.

### BACKGROUND OF THE INVENTION

T Cell Receptors

Major Histocompatibility Complex (MHC) class I gene products are widely expressed by various cell types while MHC class II molecules are expressed constitutively or are inducible in fewer, yet rather diverse cell types, such as dendritic cells, B lymphocytes, macrophages and vascular endothelial cells. By contrast, the T cell receptor complex is thought to be expressed solely by T cells, which further possess complicated signaling cascades as well as specific enzymes engaged in TCR gene rearrangement. Thus, recognition of MHC presented peptides seems to be a highly specific T cell function.

MHC-restricted T cells express heterodimeric surface protein receptors (αβTCR) that co-localize with up to five additional non-variant membrane receptors (Strominger, 1989; Abbas et al., 1994; Jameson et al., 1995). This TCR complex specifically binds processed peptide antigens associated with MHC molecules. The interactions of TCR with MHC bound peptides on various target cells may have consequences both in terms of T cell proliferation and in activation of effector mechanisms leading to target cell killing, graft rejection, and other biological effects.

Functional TCR α and β chain genes, which are capable of being expressed as polypeptides, are normally present only in cells of the T lymphocyte lineage. These functional TCR genes are formed by somatic rearrangement of germline gene segments. Each TCR locus consists of variable (V), joining (J), and constant (C) region genes, and the β chain locus contains diversity (D) gene segments. In mice there are 20 to 30 Vβ gene segments that are located 5′ of the two clusters of C and J segments. There is a single Cα gene associated with a large 5′ cluster of up to 50 different J segments and about 75 Vα and Jα exons, which includes the entire TCR δ chain locus. During maturation of T cells in the thymus, the TCR segments are rearranged in a defined order, resulting in the formation of functional TCRα and β genes in which V, D, J and C segments are in close proximity to each other.

The β chain locus rearranges prior to the α locus. The primary transcripts contain noncoding intronic sequences between the VDJ and C genes, which are later spliced out. The functional T cell receptor is comprised of 2 polypeptides: the α chain is a 40 to 60 kD acidic glycoprotein, and the β chain is a 40 to 50 kD uncharged or basic glycoprotein. The V and C regions of α and β chains form intrachain disulfide bond loops, which might contribute to the formation of a tertiary structure and are present on the cell membrane. The C region contains the transmembrane domain and a short cytoplasmic tail thought to be too small to have intrinsic signal transducing properties.

T cells (Qian et al., 1993; Yoshikai et al., 1984) as well as B cells (Calnan and Peterlin, 1986) express a series of incomplete transcripts of TCRα and β, that vary in size and structure. These transcripts may be out of frame or their sequence may contain many stop codons. In some cases mRNAs encoding the constant region flanked by an upstream spliced J segment were identified. In one case such a transcript of human TCRβ which contains an in-frame codon for methionine has been reported (Fagioli et al., 1991). However, no evidence for the existence of a protein encoded by these transcripts in T cells has been documented.

TCR transcripts have also been reported in cell lineages other than T or B-lymphocytes. Thus, TCRα mRNA was identified in murine kidney (Madrenas et al., 1991; Madrenas et al., 1992; Madrenas et al., 1994). A recent study identified in epithelial tumor cells a partial TCRγ chain mRNA, lacking the V region. This mRNA encodes a 7 kDa protein, TARP, which is translated from an alternate reading frame and is therefore not homologous to the TCRγ protein (Essand et al., 1999; Wolfgang et al., 2000). No evidence for TCRαβ or TCRδ transcripts or proteins was found in this study. It is therefore generally accepted that TCRβ transcripts are not found outside of the lymphocyte lineage and that TCR protein expressed at the cell surface is a specific T cell trait.

Mesenchymal Cells

Mesenchymal cells play a central role in embryogenesis by directing organogenesis. In the adult organism, tissue remodeling, such as that occurring in wound healing, is initiated by mesenchymal fibroblasts. The study of regulation of hemopoiesis demonstrated that blood cell formation is locally regulated by stromal mesenchyme (Zipori, 1989; Zipori et al., 1989; Zipori, 1990; Weintroub et al., 1996). Indeed, bone marrow-derived primary stroma as well as a variety of mesenchymal cells lines derived from primary bone marrow cultures exhibit the capacity to support hemopoiesis in vitro and, upon transplantation, promote the formation of bone and hemopoietically active tissue in vivo at the site of transplantation. The molecules that mediate the stromal activities have been shown to be a variety of cytokines and adhesion molecules. However, the molecules identified thus far cannot account for the wide spectrum of stromal cell functions and certainly do not explain stroma organization, stem cell renewal and other vital stromal functions.

Citation of any document herein is not intended as an admission that such document is pertinent prior art, or considered material to the patentability of any claim of the present application. Any statement as to content or a date of any document is based on the information available to the applicant at the time of filing and does not constitute an admission as to the correctness of such a statement.

### SUMMARY OF THE INVENTION

The present invention relates to novel polynucleotide transcripts and encoded proteins, which are short versions of α and β chains of the T cell receptor (TCR) as detailed herein below, and to uses of these molecules.

According to the present invention it is now disclosed that bone marrow derived stromal mesenchymal cells express unique truncated T cell receptor gene transcripts. Furthermore, these unique transcripts comprise intronic I sequences but lack variable (V) region sequences.

The present invention relates, in one aspect, to a cDNA molecule encoded by a T cell receptor (TCR) gene in non-hemopoietic cells, particularly in stromal mesenchymal cells, said cDNA molecule lacking V region sequences and comprising a constant (C) domain and joining (J) region sequences, and a 5′ intronic J sequence upstream to said J region sequence including an in-frame methionine codon.

The novel polynucleotide sequences disclosed herein and the corresponding proteins, polypeptides or peptides encoded by these polynucleotide sequences may be derived from any mammalian species including human genetic material.

In certain embodiments of the invention, the cDNA molecule is encoded by a mouse TCRβ gene. The joining (J) gene sequence may be selected from, but is not limited to, Jβ2.1 and Jβ2.6.

According to one embodiment of the invention, the joining (J) gene sequence may be Jβ2.1 and said 5′ intronic J sequence including an in-frame methionine codon encodes a peptide of the sequence M E N V S N P G S C I E E G E E R G R I L G S P F L [SEQ ID NO:1].

In an alternative embodiment, the joining (J) gene sequence is Jβ2.6 and said 5′ intronic J sequence including an in-frame methionine codon codes for a peptide of the sequence M G E Y L A E P R G F V C G V E P L C [SEQ IDNO:2].

In another embodiment of the invention, the cDNA molecule is encoded by a mouse TCRα gene. In this case, the joining (J) gene sequences are selected from, but not limited to, JαTA31, JαTA46, JαNew05, JαS58, JαNew06, JαNew08, JαLB2A, JαDK1, and JαTA39.

According to this embodiment of the invention, the cDNA molecule comprises a 5′ intronic J sequence including an in-frame methionine codon selected from the group consisting of:

(i) the intronic JαTA31 gene sequence coding for the peptide:

| | |
|---|---|
| MAWH; | [SEQ ID NO:3] |

(ii) the intronic JαTA46 gene sequence coding for the peptide:

| | |
|---|---|
| MEAGWEVQHWVSDMECLTV; | [SEQ ID NO:4] |

(iii) the intronic JαTA46 gene sequence coding for the peptide:

| | |
|---|---|
| MECLTV; | [SEQ ID NO:5] |

(iv) the intronic JαNew05 gene sequence coding for the peptide:

| | |
|---|---|
| MTV; | [SEQ ID NO:6] |

(v) the intronic JαS58 gene sequence coding for the peptide:

| | |
|---|---|
| MCGSEEVFVVESA; | [SEQ ID NO:7] |

(vi) the intronic JαNew06 gene sequence coding for the peptide:

| | |
|---|---|
| MACYQMYFTGRKVDEPSELGSGL | [SEQ ID NO:8] |
| ELSYFHTGGSSQAVGLFIENMIST | |
| SHFHFQEMQFSIWSFTVLQISAPG | |
| SHLVPETERAEGPGVFVEHDI; | |

(vii) the intronic JαNew06 gene sequence coding for the peptide:

| | |
|---|---|
| MYFTGRKVDEPSELGSGLELSYFH | [SEQ ID NO:9] |
| TGGSSQAVGLFIENMISTSHGHFQE | |
| MQFSIWSFTVLQISAPGSHLVPETE | |
| RAEGPGVFVEHDI; | |

(viii) the intronic JαNew06 gene sequence coding for the peptide:

| | |
|---|---|
| MISTSHGHFQEMQFSLWSFTVLQIS | [SEQ ID NO:10] |
| APGSHLVPETERAEGPGVFVEHDI; | |

(ix) the intronic JαNew06 gene sequence coding for the peptide:

| | |
|---|---|
| MQFSIWSFTVLQISAPGSH | [SEQ ID NO:11] |
| LVPETERAEGPGVFYEHDI; | |

(x) the intronic JαNew08 gene sequence coding for the peptide:

| | |
|---|---|
| MWWGLILSASVKFLQRKEILC; | [SEQ ID NO:12] |

(xi) the intronic JαLB2A gene sequence coding for the peptide:

| | |
|---|---|
| MVGADLCKGGWHCV; | [SEQ ID NO:13] |

(xii) the intronic JαDK1 gene sequence coding for the peptide:

| | |
|---|---|
| MREPVKNLQGLVS; | [SEQ ID NO:14] |

(xiii) the intronic JαTA39 gene sequence coding for the peptide:

M E V Y E L R V T L M E T G R E R S H F V K T S L [SEQ ID NO:15]; and

(xiv) the intronic JαTA39 gene sequence coding for the peptide:

| | |
|---|---|
| METGRERSHFVKTSL. | [SEQ ID NO: 16] |

According to an alternative and more preferred embodiment, the novel intronic sequences and their corresponding peptides may be derived from human genetic material. Any known sequences, such as intronic sequences of the joining segment of human Jβ2.3 gene known in tumor cells (Kimoto, 1998) are explicitly excluded from the claimed novel sequences.

According to an embodiment of the invention, the cDNA molecule comprises a 5′ intronic J sequence including an in-frame methionine codon consisting of the human intronic Jβ2.3 gene sequence coding for the peptide M G L S A V G R T R A E S G T A E R A A P V F V L G L Q A V [SEQ ID NO:17].

In another embodiment of the invention, the cDNA molecule is encoded by a human TCRα gene. In this case, the joining (J) gene sequences are selected from, but not limited to, Jα2, Jα3, Jα6, Jα8, Jα9, Jα11, Jα13, Jα14, Jα24, Jα25, Jα31, Jα36, Jα40, Jα41 and Jα44.

According to additional embodiments of the invention, the cDNA molecule comprises a 5′ intronic J sequence, including an in-frame methionine codon selected from group consisting of:

1) the intronic Jα2 gene sequence coding for an in-frame M (It will be appreciated by the skilled artisan that this amino acid will not appear as an isolated amino acid residue but rather refers to a single in frame methionine encoded by the intronic sequence which is part of the larger TCR molecule (J and C regions) described above and which is transcribed in the novel transcripts of the invention.)

2) the intronic Jα3 gene sequence coding for the peptide:

| | |
|---|---|
| MLLWDPSGFQQISIKKVISKTLPT; | [SEQ ID NO:18] |

3) the intronic Jα6 gene sequence coding for the peptide:

| | |
|---|---|
| MLPNTMGQLVEGGHMKQVLSKAVLTV; | [SEQ ID NO:19] |

4) the intronic Jα6 gene sequence coding for the peptide:

| | |
|---|---|
| MGQLVEGGHMKQVLSKAVLTV; | [SEQ ID NO:20] |

5) the intronic Jα6 gene sequence coding for the peptide:

| | |
|---|---|
| MKQVLSKAVLTV; | [SEQ ID NO:21] |

6) the intronic Jα8 gene sequence coding for the peptide:

| | |
|---|---|
| MSEC; | [SEQ ID NO:22] |

7) the intronic Jα9 gene sequence coding for the peptide:

| | |
|---|---|
| MAHFVAVQITV; | [SEQ ID NO:23] |

8) the intronic Jα11 gene sequence coding for the peptide:

| | |
|---|---|
| MGICYS; | [SEQ ID NO :24] |

9) the intronic Jα 13 gene sequence coding for the peptide:

| | |
|---|---|
| MKRAGEGKSFCKGRHYSV; | (SEQ ID NO:25] |

10) the intronic Jα14 gene sequence coding for the peptide:

| | |
|---|---|
| MLTTLIYYQGNSVIFVRQHSA; | [SEQ ID NO:26] |

11) the intronic Jα24 gene sequence coding for the peptide:

| | |
|---|---|
| MQLPHFVARLFPHEQFVFIQQLSSLGKPFCRGVCH | [SEQ ID NO:27] |
| SV; | |

12) the intronic Jα25 gene sequence coding for the peptide: M (see comment in item 1 above)

13) the intronic Jα31 gene sequence coding for the peptide:

| | |
|---|---|
| MGFSKGRKCCG; | [SEQ ID NO:28] |

14) the intronic Jα36 gene sequence coding for the peptide:

| | |
|---|---|
| MKKIWLSRKVFLYWAETL; | [SEQ ID NO:29] |

15) the intronic Jα40 gene sequence coding for the peptide:

| | |
|---|---|
| MGKVHVMPLLFMESKAASINGNIMLVYVETHNTV; | [SEQ ID NO:30] |

16) the intronic Jα40 gene sequence coding for the peptide:

| | |
|---|---|
| MPLLFMESKAASINGNIMLVYVETHNTV; | [SEQ ID NO:31] |

17) the intronic Jα40 gene sequence coding for the peptide:

| | |
|---|---|
| MESKAASINGNIMLVYVETHNTV; | [SEQ ID NO:32] |

18) the intronic Jα 40 gene sequence coding for the peptide:

| | |
|---|---|
| MLVYVETHNTV; | [SEQ ID NO:33] |

19) the intronic Jα41 gene sequence coding for the peptide:

| | |
|---|---|
| MEEGSFIYTIKGPWMTHSLCDCCVIGFQTLALI | [SEQ ID NO:34] |
| GIIGEGTWWLLQGVFCLGRTHC; | |

20) the intronic Jα41 gene sequence coding for the peptide:

| | |
|---|---|
| M T H S L C D C C V I G F Q T L A L | [SEQ ID NO:35] |
| I G I I G E G T W W L L Q G V F C L | |
| G R T H C; | |

21) the intronic Jα44 gene sequence coding for the peptide:

| | |
|---|---|
| MESQATGFCYEASHSV. | [SEQ ID NO:36] |

In another aspect, the invention relates to antisense DNA molecules of any of the cDNA molecules of the invention described above.

The invention further relates to expression vectors comprising the cDNA and antisense molecules of the invention, and to host cells, particularly mammalian cells, comprising said vectors. In one preferred embodiment the host cells are transfected mesenchymal human cells.

The cDNA of the invention can be used to transfect mesenchymal human cells for inducing mesenchymal cell growth. Thus the invention relates to compositions comprising said transfected mesenchymal human cells for use in disorders requiring induction of mesenchymal cell growth, such as wound healing.

The invention further relates to a method for inducing mesenchymal cell growth comprising the step of administering to a subject in need thereof transfected mesenchymal human cells comprising a cDNA molecule according to the invention, in an amount effective to induce mesenchymal cell growth. This method is preferably applicable for enhanced wound healing.

The antisense DNA molecules of the invention can be used to transfect mesenchymal human cells for inhibiting or suppressing mesenchymal cell growth. Thus the invention relates to compositions comprising said transfected mesenchymal human cells for use in disorders requiring inhibition or suppression of mesenchymal cell growth, such as in carcinomas.

The invention further relates to a method for suppressing mesenchymal cell growth comprising the step of administering to a subject in need thereof an antisense DNA molecule of the invention and/or autologous transfected mesenchymal human cells comprising an antisense DNA molecule of the invention, in an amount effective to suppress mesenchymal cell growth, such as for suppression of carcinomas.

The invention further relates to a polypeptide encoded by a polynucleotide of the invention. In one embodiment, said polypeptide is a protein capable of being expressed in mesenchymal cells, either on the cell surface or intracellularly. In one exemplary embodiment the polynucleotide is encoded by the nucleotide sequence depicted in FIG. 1 and the polypeptide comprises the amino acid sequence depicted in FIG. 1.

The invention still further relates to a synthetic peptide deduced from an intronic J sequence of a TCR.

Examples of such peptides derived from non-human animals include but are not limited to:

| | | |
|---|---|---|
| (a) | MENVSNPGSCIEEGEERGRILGSPFL; | [SEQ ID NO:1] |
| (b) | MGEYLAEPRGFVCGVEPLC; | [SEQ ID NO:2] |
| (c) | MAWH; | [SEQ ID NO:3] |
| (d) | MEAGWEVQHWVSDMEGLTV; | [SEQ ID NO:4] |
| (e) | MECLTV; | [SEQ ID NO:5] |
| (f) | MTV; | [SEQ ID NO:6] |
| (g) | MCGSEEVFVVESA; | [SEQ ID NO:7] |
| (h) | MACYQMYFTGRKVDEPSELGSGL | [SEQ ID NO:8] |
| | ELSYFHTGGSSQAVGLFIENMIST | |
| | SHGHFQEMQFSIWSFTVLQISAPG | |
| | SHLVPETERAEGPGVFVEHDI; | |
| (i) | MYFTGRKVDEPSELGSGLELSYFH | [SEQ ID NO:9] |
| | TGGSSQAVGLFIENMISTSHGHFQE | |
| | MQFSIWSFTVLQISAPGSHLVPETE | |
| | RAEGPGVFVEHDI; | |
| (j) | MISTSHGHFQEMQFSIWSFTVLQIS | [SEQ ID NO:10] |
| | APGSHLVPETERAEGPGVFVEHDI; | |
| (k) | MQFSIWSFTVLQISAPGSH | |
| | LVPETERAEGPGVFVEHDI; | [SEQ ID NO:11] |
| (l) | MWWGLILSASVKFLQRKEILC; | [SEQ ID NO:12] |
| (m) | MVGADLCKGGWHCV; | [SEQ ID NO:13] |
| (n) | MREPVKNLQGLVS; | [SEQ ID NO:14] |
| (o) | MEVYELRVTLMETGRERSHFVKTSL; | [SEQ ID NO:15] |
| | and | |
| (p) | METGRERSHFVKTSL. | [SEQ ID NO:16] |

Examples of useful peptides according to the present invention derived from human sources include but are not limited to:

| | | |
|---|---|---|
| i) | MGLSAVGRTRAESGTAERAAPV | [SEQ ID NO:17] |
| | FVLGLQAV; | |
| ii) | MLLWDPSGFQQISIKKVISKTL | [SEQ ID NO:18] |
| | PT; | |
| iii) | MLPNTMGQLVEGGHMKQVLSKA | [SEQ ID NO:19] |
| | VLTV; | |
| iv) | MGQLVEGGHMKQVLSKAVLTV; | [SEQ ID NO:20] |
| v) | MKQVLSKAVLTV; | [SEQ ID NO:21] |
| vi) | MSEC; | [SEQ ID NO:22] |
| vii) | MAHFVAVQITV; | [SEQ ID NO:23] |
| viii) | MGICYS; | [SEQ ID NO:24] |
| ix) | MKRAGEGKSFCKGRHYSV; | (SEQ ID NO:25] |
| x) | MLTTLIYYQGNSVIFVRQHSA; | [SEQ ID NO:26] |
| xi) | MQLPHFVARLFPHEQFVFIQQL | [SEQ ID NO:27] |
| | SSLGKPFCRGVCHSV; | |
| xii) | MGFSKGRKCCG; | [SEQ ID NO:28] |
| xiii) | MKKIWLSRKVFLYWAETL; | [SEQ ID NO:29] |
| xiv) | MGKVHVMPLLFMESKAASINGN | [SEQ lID NO:30] |
| | IMLVYVETHNTV; | |
| xv) | MPLLFMESKAASINGNIMLVYV | [SEQ ID NO:31] |
| | ETHNTV; | |
| xvi) | MESKAASINGNIMLVYVETHNT | [SEQ ID NO:32] |
| | V; | |
| xvii) | MLVYVETHNTV; | [SEQ ID NO:33] |
| xviii) | MEEGSFIYTIKGPWMTHSLCDC | [SEQ ID NO:34] |
| | CVIGFQTLALIGIIGEGTWWLL | |
| | QGVFCLGRTHC; | |
| xix) | MTHSLCDCCVIGFQTLALIGII | [SEQ ID NO:35] |
| | GEGTWWLLQGVFCLGRTHC; | |
| | and | |
| xx) | MESQATGFCYEASHSV. | [SEQ ID NO:36] |

In still a further aspect, the invention relates to an antibody that binds to a synthetic peptide having a sequence encoded by intronic sequences of the TCR genes. According to one preferred embodiment the antibodies bind to a synthetic peptide having the sequence LAEPRGFVCGVE [SEQ ID NO:37]. These antibodies are useful as markers of mesenchymal cells, for example for diagnostic purposes and for prognosis of cancer.

### BRIEF DESCRIPTION OF THE DRAWINGS

FIG. 1 depicts the nucleotide sequence of the J^{int}J-Cβ₂ mRNA transcript of the stromal/mesenchymal cell line [SEQ ID NO:38], MBA-13, and the deduced amino acid sequence encoded thereby [SEQ ID NO:39]. The cDNA products were obtained from reverse transcription (RT)-PCR analysis using TCR primers and sequenced.

FIGS. 2A-2F show flow cytometric analysis of J^{int}J-Cβ₂ expression by mesenchymal cells. Mouse embryonic fibroblasts (MEF) (2E) and different MBA-13 cell strains (1-3; 2A-2C, respectively) were stained with preimmunized (histogram I) or immunized (histogram II) purified antibodies from rabbit serum. The rabbits were immunized with a synthetic segment of SEQ ID NO:2, namely SEQ ID NO:37, with the sequence LAEPRGFVCGVE. As a second antibody, we used Fab FITC conjugated donkey anti-rabbit IgG. Staining with second antibody only gave a histogram shown in histogram III. Cells stained with rabbit polyclonal antibodies to irrelevant peptide 1121 of the sequence RGGGGGRGGLHD, similarly produced and purified, served as negative control (histogram IV). Competition of antibody binding was performed by pre-incubation of the purified immune serum with the specific immunizing peptide SEQ ID NO:37 for 30 min at room temperature (2D, histogram). Competition with irrelevant peptide 1121 served as negative control (data not shown). The results of one experiment, out of three performed, are shown.

FIG. 3 shows RT-PCR analysis of the novel TCRCβ2 cDNA including an in-frame intronic J sequence designated J^{int}J-Cβ₂, obtained from MBA-13 mesenchymal cell line and fetal primary cell cultures. The cDNA was obtained from total RNA extracted from mouse embryonic fibroblast and different MBA-13 cell strains (1-3). RT-PCR was performed using the following sense pairs:

exonic Jβ2.6:
5′-CTATGAACAGTACTTCGGTC-3′;
or
intronic Jβ2.6:
5′-ATGGGAGAATACCTCGCTG-3′;
or
5′-CCCTAAATGGGAGAATACC;
and
antisense primer Cβ3:
5′-CATCCTATCATCAGGGGGTTCTGTCTGCAA-3′.

Products of 465 bp and 524 bp were produced, respectively.

FIG. 4 depicts sequences of all possible versions of mouse TCRαβ containing an intronic 5′ end including an in-frame Met codon as collected from available data bases: the intronic Jβ sequences Jβ2.1 and Jβ2.6, and the intronic Jα sequences JαTA31, JαTA46, JαNew05, JαS58, JαNew06, JαNew08, Jα LB2A, JαDK1 and JαTA39.

FIG. 5 depicts sequences of all possible versions of the human TCRαβ containing an intronic 5′ end including an in-frame Met codon as collected from available data bases: the intronic Jβ sequence Jβ2.3, and the intronic Jα sequences Jα2, Jα3, Jα6, Jα8, Jα9, Jα11, Jα13, Jα14, Jα24, Jα25, Jα31, Jα36, Jα40, Jα41 and Jα44.

FIG. 6 shows determination of generation time of different clones of MBA-13 cell line. Eight individual clones were studied by PCR for expression of M-TCR (TCRβ J^{int}-J_{2.6}C). Out of those, four were found to be negative (M-TCR⁻ clones E4, C6, G1, B7) and four were found to be positive (M-TCR⁺ clones C4, D10, B10, B1). Cells were seeded at different concentrations (10³, 5×10³ and 10⁴/ml) and cell growth was determined after 44-46 hours. The population generation time was calculated.

FIGS. 7A-7C show RT-PCR analysis of TCR expression in different cell lines and primary cell cultures. cDNA was obtained from total RNA extracted from different cell types, as described in the Materials and Methods section hereinafter, and RT-PCR was performed using the following primer pairs: Cβ1 and Cβ2 primers for TCRCβ2 produced a 410 bp product (FIG. 7A); Cα1 and Tm or Cα1 and Cα2 for TCRCα produced a 356 bp or 138 bp product, respectively (FIGS. 7B and 7C).

FIGS. 8A-8D show mRNA expression of TCRCβ (8A-8B), TCRCα (8C) and CD3ε (8D) mRNA transcripts. Poly A+mRNA, from mesenchymal (MBA-13, AC-6, NIH3T3, thymus and MEF), epithelial (1C8) and endothelial-adipocyte (14F1.1) cell lines, was Northern blotted as described in the Materials and Methods section hereinafter, and probed with the following probes: TCRCβ, TCRCα and CD3ε. For the TCRCβ chain, thymus RNA exhibited 1.3 kb (full-length) and 1.0 kb (truncated) transcripts, while the mesenchymal MBA-13, AC-6 and MEF cells exhibited a 1.1 kb transcript (FIGS. 8A and 8B). For the TCRCα chain, thymus RNA and non-T cell lines exhibited a 1.6 kb transcript (FIG. 8C). For the CD3ε chain, thymus RNA exhibited a 1.5 kb transcript, while non-T cells showed a transcript whose size was slightly larger (FIG. 8D). Hybridization signals for TCRCβ were quantitated by densitometric scanning, and the signal value of MBA-13 was 60 fold less than thymocytes.

FIG. 9 shows flow cytometric analysis of CD3ε, TCRαβ and TCRγδ antigen expression by MBA-13 cells. MBA-13 cells were stained with FITC-conjugated TCRαβ, CD3ε and with PE-conjugated TCRγδ (solid line). For intracellular staining, cells were fixed and stained with FITC-conjugated TCRαβ using the Cytoperm kit. In all experiments, cells stained with isotype-matched FITC-conjugated rat anti-mouse IgG were also prepared as negative controls (dotted line). The results of a single experiment are shown.

FIG. 10 Detection of a mesenchymal cell surface antigen reactive with an anti-TCRβ antibody. Flow cytometric analysis of MEF from wild type (solid black line) or from TCR^{√-} mutant mice (***solid grey line) stained by the FITC-conjugated hamster anti-mouse TCRβ H57-597 monoclonal antibodies. The dotted line indicates the isotype control.

FIG. 11 Human TCR Jβ2.3-Cβ. transcript cloned from cDNA of cord blood mononuclear cells and amniotic fluid cells. The cloned transcripts were sequenced and were found to be identical. The lines above the sequence indicate the boundaries of each segment The predicted protein product is shown below the sequence. Bold font indicates an A to G transition that was found in both clones.

FIG. 12 Expression of GFP-TCR Jβ2.3-Cβ in 293T transfected cells. Western blot analysis. Each lane was loaded with lysate of 5×10⁵ cells, GFP-TCR Jβ2.3-Cβ was detected with Anti-GFP monoclonal antibody JL-8.

FIG. 13 Recombinant mesenchymal TCRβ (GFP-J^{int}-Jβ2.6-C) in a preTCR-like complex causes apoptotic cell death upon overexpression. (A) Immunofluorescence analysis of cells transfected with cDNA constructs encoding a fusion protein of J^{int}-Jβ2.6-C linked to GFP, together with pTα HA vector. (B) Western blot analysis of extracts from 293T cells transfected with GFP-J^{int}-Jβ2.6-C together with HA-pTα (lane 1). Control GFP and HA vectors (lane 2), GFP vector and HA-pTα0 (lane 3), HA vector and GFP-J^{int}-Jβ2.6-C (lane 4) and untransfected cells (lane 5). Immunoblotting was performed with an anti-GFP monoclonal antibody. The position of the fusion protein, GFP-J^{int}-Jβ2.6-C is indicated (GFP-J^{int}) as is the position of GFP free protein (GFP). (C) Cell cycle flow cytometric analysis of 293T cells transfected with the indicated vectors. The cell cycle analysis of GFP negative cells that were treated with GFP-J^{int}-Jβ2.6-C and pTα but remained untransfected (C-I) serves as a representative control; similar patterns were observed following transfection with empty vectors or separately with GFP-J^{int}-Jβ2.6-C and pTα.

FIG. 14 Properties of individual clones of the MBA-13 cell line in which tumor formation of MBA-13 clones expressing high (D10, B10, C4) or low (C6, B7) TCRβ was examined following intradermal injection into nude CD1 mice at 10⁶ cells per site.

### DETAILED DESCRIPTION OF THE INVENTION

I. Truncated T Cell Receptor mRNA and Protein Expression

The present invention relates to new mRNA transcripts and proteins encoded by these transcripts which are short versions of α and β TCR as detailed and to uses of these molecules.

While studying the interactions of stromal cell lines with thymic T cells, we used reverse transcription polymerase chain reaction (RT-PCR) to amplify TCR gene fragments. Unexpectedly, the MBA-13 mesenchymal stromal cell line, derived from mouse bone marrow, was found to consistently express TCRβ constant (Cβ) region, while cDNA from a negative control tissue, i.e. liver, and from several control cell lines such as pre-B cells, plasmacytoma and mastocytoma cells, did not produce PCR products using primers from the TCR gene.

Further studies with a variety of stromal cell lines, in accordance with the present invention, showed the existence of TCR gene derived mRNAs that encode truncated versions of the TCR consisting of the constant (C) domain, which is identical to that of T cell receptor, a joining (J) region, which may be one of several alternatives, and a 5′ domain consisting of a nucleotide sequence corresponding to an intronic J sequence (again one of several alternatives) including an in-frame codon for methionine. This mRNA lacks V region sequences. One of such molecules, namely a new version of a TCRβ2.6, is shown herein to exist in mesenchymal cells and to encode a cell surface mesenchyrnal protein. Expression on the mRNA level has also been observed in the thymus. Identification of this stromal cell surface TCR-like antigen, by H57-597 antibodies, was further demonstrated in MEF from wild type mice, whereas no similar antigen was observed in MEF from TCRβ^{√-} mutant mice, that did not express J^{int}-Jβ2.6-C mRNA, providing genetic support for the existence of this TCR protein in mesenchymal cells.

We further disclose that these novel truncated TCR variants are functionally involved in mesenchymal cell growth.

II. Antisense Sequences

As will be exemplified herein below, the expression or lack of expression of the mesenchymal TCR seems to control mesenchymal cell growth. The invention therefore further relates to the use of the cDNA and antisense molecules of the invention derived from mesenchymal TCR mRNAs for expression in cells and tissues for the purpose of modulating stromal/mesenchymal cell growth.

For this purpose, the cDNA or antisense molecule is inserted in appropriate vectors such as, but not limited to, the retroviral vectors DCAl and DCMm that have been used in clinical trials in gene therapy (Bordignon et al., 1995). Preferably, the vector containing the cDNA or the antisense molecule, under the control of a suitable promoter such as that cDNA's own promoter, will be used to infect or transfect suitable mammalian, preferably human, most preferably the patient's autologous mesenchymal cells. The genetically modified mesenchymal cells are then administered to a patient in need thereof by an appropriate route and are expressed in the desired site or tissue.

In order to manipulate the expression of an undesirable gene, it is necessary to produce antisense RNA in a cell. To this end, the complete or partial cDNA of an undesirable gene in accordance with the present invention is inserted into an expression vector comprising a promoter. The 3′ end of the cDNA is thereby inserted adjacent to the 3′ end of the promoter, with the 5′ end of the cDNA being separated from the 3′ end of the promoter by said cDNA. Upon expression of the cDNA in a cell, an antisense RNA is therefore produced which is incapable of coding for the protein. The presence of antisense RNA in the cell reduces the expression of the cellular (genomic) copy of the undesirable gene.

For the production of antisense RNA, the complete cDNA may be used. Alternatively, a fragment thereof may be used, which is preferably between about 9 and 1,000 nucleotides in length, more preferably between 15 and 500 nucleotides, and most preferably between 30 and 150 nucleotides.

The fragment is preferably corresponding to a region within the 5′ half of the cDNA, more preferably the 5′ region comprising the 5′ untranslated region and/or the first exon region, and most preferably comprising the ATG translation start site. Alternatively, the fragment may correspond to DNA sequence of the 5′ untranslated region only.

A synthetic oligonucleotide may be used as antisense oligonucleotide. The oligonucleotide is preferably a DNA oligonucleotide. The length of the antisense oligonucleotide is preferably between 9 and 150, more preferably between 12 and 60, and most preferably between 15 and 50 nucleotides. Suitable antisense oligonucleotides that inhibit the production of the protein of the present invention from its encoding mRNA can be readily determined with only routine experimentation through the use of a series of overlapping oligonucleotides similar to a “gene walking” technique that is well-known in the art. Such a “walking” technique as well known in the art of antisense development can be done with synthetic oligonucleotides to walk along the entire length of the sequence complementary to the mRNA in segments on the order of 9 to 150 nucleotides in length. This “gene walking” technique will identify the oligonucleotides that are complementary to accessible regions on the target mRNA and exert inhibitory antisense activity.

Alternatively, an oligonucleotide based on the coding sequence of a protein capable of binding to an undesirable gene or the protein encoded thereby can be designed using Oligo 4.0 (National Biosciences, Inc.). Antisense molecules may also be designed to inhibit translation of an mRNA into a polypeptide by preparing an antisense which will bind in the region spanning approximately −10 to +10 nucleotides at the 5′ end of the coding sequence.

Modifications of oligonucleotides that enhance desired properties are generally used when designing antisense oligonucleotides. For instance, phosphorothioate bonds are used instead of the phosphoester bonds that naturally occur in DNA, mainly because such phosphorothioate oligonucleotides are less prone to degradation by cellular enzymes. Preferably, 2′-methoxyribonucleotide modifications in 60% of the oligonucleotide is used. Such modified oligonucleotides are capable of eliciting an antisense effect comparable to the effect observed with phosphorothioate oligonucleotides.

Therefore, the preferred antisense oligonucleotide of the present invention has a mixed phosphodiester-phosphorothioate backbone. Preferably, 2′-methoxyribonucleotide modifications in about 30% to 80%, more preferably about 60%, of the oligonucleotide are used.

In the practice of the invention, antisense oligonucleotides or antisense RNA may be used. The length of the antisense RNA is preferably from about 9 to about 3,000 nucleotides, more preferably from about 20 to about 1,000 nucleotides, most preferably from about 50 to about 500 nucleotides.

In order to be effective, the antisense oligonucleotides of the present invention must travel across cell membranes. In general, antisense oligonucleotides have the ability to cross cell membranes, apparently by uptake via specific receptors. As the antisense oligonucleotides are single-stranded molecules, they are to a degree hydrophobic, which enhances passive diffusion through membranes. Modifications may be introduced to an antisense oligonucleotide to improve its ability to cross membranes. For instance, the oligonucleotide molecule may be linked to a group which includes a partially unsaturated aliphatic hydrocarbon chain and one or more polar or charged groups such as carboxylic acid groups, ester groups, and alcohol groups. Alternatively, oligonucleotides may be linked to peptide structures, which are preferably membranotropic peptides. Such modified oligonucleotides penetrate membranes more easily, which is critical for their function and may, therefore, significantly enhance their activity.

III. Introduction of Proteins, Peptides, and DNA into Cells

The present invention provides proteins encoded by the truncated TCR genes, peptides derived therefrom and antisense DNA molecules based on the TCR transcripts. A therapeutic or research-associated use of these tools necessitates their introduction into cells of a living organism or into cultured cells. For this purpose, it is desired to improve membrane permeability of peptides, proteins and antisense molecules. The same principle, namely, derivatization with lipophilic structures, may also be used in creating peptides and proteins with enhanced membrane permeability. For instance, the sequence of a known membranotropic peptide may be added to the sequence of the peptide or protein. Further, the peptide or protein may be derivatized by partly lipophilic structures such as the above-noted hydrocarbon chains, which are substituted with at least one polar or charged group. For example, lauroyl derivatives of peptides have been described in the art. Further modifications of peptides and proteins include the oxidation of methionine residues to thereby create sulfoxide groups and derivatives wherein the relatively hydrophobic peptide bond is replaced by its ketomethylene isoester (COCH₂) have been described. It is known to those of skill in the art of protein and peptide chemistry these and other modifications enhance membrane permeability.

Another way of enhancing membrane permeability is to make use of receptors, such as virus receptors, on cell surfaces in order to induce cellular uptake of the peptide or protein. This mechanism is used frequently by viruses, which bind specifically to certain cell surface molecules. Upon binding, the cell takes the virus up into its interior. The cell surface molecule is called a virus receptor. For instance, the integrin molecules CAR and AdV have been described as virus receptors for Adenovirus. The CD4, GPR1, GPR15, and STRL33 molecules have been identified as receptors/coreceptors for HV.

By conjugating peptides, proteins or oligonucleotides to molecules that are known to bind to cell surface receptors, the membrane permeability of said peptides, proteins or oligonucleotides will be enhanced. Examples of suitable groups for forming conjugates are sugars, vitamins, hormones, cytokines, transferrin, asialoglycoprotein, and the like molecules. Low et al U.S. Pat. No. 5,108,921 describes the use of these molecules for the purpose of enhancing membrane permeability of peptides, proteins and oligonucleotides, and the preparation of said conjugates.

Low and coworkers further teach that molecules such as folate or biotin may be used to target the conjugate to a multitude of cells in an organism, because of the abundant and nonspecific expression of the receptors for these molecules.

The above use of cell surface proteins for enhancing membrane permeability of a peptide, protein or oligonucleotide of the invention may also be used in targeting the peptide, protein or oligonucleotide of the present invention to certain cell types or tissues. For instance, if it is desired to target neural cells, it is preferable to use a cell surface protein that is expressed more abundantly on the surface of those cells.

The protein, peptide or oligonucleotide of the invention may therefore, using the above-described conjugation techniques, be targeted to mesenchymal cells. For instance, if it is desired to enhance mesenchymal cell growth in order to augment autologous or allogeneic bone marrow transplantation or wound healing, then the TCR variant gene could be inserted into mesenchymal cells as a form of gene therapy. In this embodiment, local application of the cells containing the cDNA molecule can be used to induce mesenchymal cell growth thus enhancing the wound healing process

In contrast, if it is desired to inhibit mesenchymal cell growth, as in the case of a tumor. Therefore, mesenchymal cells of the tumor can be transfected with the antisense cDNA and then be used for treatment of localized solid tumors, to achieve regression of the tumor mesenchyme and subsequent regression of the tumor.

The proteins encoded by the mRNAs of the invention are cell surface receptors of mesenchymal cells and may probably interact with ligands presented by neighboring hemopoietic or non-hemopoietic cells. Thus, in bound or soluble form, these proteins or the peptides derived therefrom, may have modulatory effects on cells that bear said ligands.

IV. Antibodies

The present invention also comprehends antibodies specific for the proteins encoded by the truncated TCR transcripts which is part of the present invention as discussed above. The proteins and peptides of the invention may be used as immunogens for production of antibodies that may be used as markers of mesenchymal cells. Such an antibody may be used for diagnostic purposes to identify the presence of any such naturally occurring proteins. Such antibody may be a polyclonal antibody or a monoclonal antibody or any other molecule that incorporates the antigen-binding portion of a monoclonal antibody specific for such a protein. Such other molecules may be a single-chain antibody, a humanized antibody, an F(ab) or F(ab′)₂ fragment, a chimeric antibody, an antibody to which is attached a label, such as fluorescent or radioactive label, or an immunotoxin in which a toxic molecule is bound to the antigen binding portion of the antibody. The examples are intended to be non-limiting. However, as long as such a molecule includes the antigen-binding portion of the antibody, it will be expected to bind to the protein and, thus, can be used for the same diagnostic purposes for which a monoclonal antibody can be used.

V. Pharmaceutical Compositions

These compositions are for use by injection, topical administration, or oral uptake. Preferred uses of the pharmaceutical compositions of the invention by injection are subcutaneous injection, intravenous injection, and intramuscular injection. Less convenient routes of administration may include intraperitoneal, intradural, intra-thecal administration or intra-arterial administration when required.

The pharmaceutical composition of the invention generally comprises a buffering agent, an agent which adjusts the osmolarity thereof, and optionally, one or more carriers, excipients and/or additives as known in the art, e.g., for the purposes of adding flavors, colors, lubrication, or the like to the pharmaceutical composition.

Carriers are well known in the art and may include starch and derivatives thereof, cellulose and derivatives thereof, e.g., microcrystalline cellulose, xanthan gum, and the like. Lubricants may include hydrogenated castor oil and the like.

A preferred buffering agent is phosphate-buffered saline solution (PBS), which solution is also adjusted for osmolarity.

A preferred pharmaceutical formulation is one lacking a carrier. Such formulations are preferably used for administration by injection, including intravenous injection.

The preparation of pharmaceutical compositions is well known in the art and has been described in many articles and textbooks.

Additives may also be selected to enhance uptake of the antisense oligonucleotide across cell membranes. Such agents are generally agents that will enhance cellular uptake of double-stranded DNA molecules. For instance, certain lipid molecules have been developed for this purpose, including the transfection reagents DOTAP (Boehringer Mannheim), Lipofectin, Lipofectam, and Transfectam, which are available commercially. The antisense oligonucleotide of the invention may also be enclosed within liposomes.

The preparation and use of liposomes, e.g., using the above-mentioned transfection reagents, is well known in the art. Other methods of obtaining liposomes include the use of Sendai virus or of other viruses.

The above-mentioned cationic or nonionic lipid agents not only serve to enhance uptake of oligonucleotides into cells, but also improve the stability of oligonucleotides that have been taken up by the cell.

Having now generally described the invention, the same will be more readily understood through reference to the following examples, which is provided by way of illustration and are not intended to be limiting of the present invention.

### EXAMPLES

Human Cell Culture

293T cell line were grown in Dulbecco's modified Eagles medium (DMEM) supplemented with 10% fetal calf serum (FCS) Beth Haemek, Israel), 20 mM L-glutamine, 60 μg/ml penicillin, 100 μg/ml streptomycin and 50 mg/L Kanamycin. Amniotic fluid cells were grown in AMF medium (Biological industries, Beit Haemek, Israel).

GFP-TCRJ2.3-Cβ Expression Vector

The cDNA of human TCR Jβ2.3-Cβ was amplified from cDNA from amniotic fluid cells and from cord blood mononuclear cells using the sense primer 5≧CCGGAATTCCATGGGGCTCTCAGCGGTGG and antisense primer 5′CGCGGA TCCCTAGCCTCTGGAATCCTTTCTC and ligated into EcoRI and BamHI digested and calf intestinal alkaline phosphatase-treated pEGFPC1 (Clontech, Palo Alto, Calif.). DNA sequence analysis of the GFP-TCR Jβ2.3-Cβ confirmed the intended reading frame. Proceeding from the N to C terminus, the resulting fusion protein consists of GFP, a linker sequence of 10 amino acids, and TCR J2.3-Cβ.

Transfections

293T cells were plated at 70% confluency in 6 well plates and transfected with 1.6 μg of GFP-TCR Jβ2.3-Cβ construct using the calcium phosphate transfection method.

Western Blot Analysis and Fluorescence Analysis

For immunoblot analysis, 24 hrs after transfection, 5×10⁵ 293T cells were lyzed on ice in Tris pH 8 20 mM containing 1% Triton, 140 mM NaCl, 10% glycerol, 1 mM EGTA, 1.5 mM MgCl₂, and 1 mM sodium vanadate. Cell lysates were clarified by centrifugation at 15,000 g for 10 min at 4° C., and boiled after addition of SDS-sample buffer (5% glycerol, 2% SDS, 62.5 mM Tris-HCL pH 6.8, 2% 2-mercaptoethanol, 0.01% bromophenol blue).

Extracts were subjected to 12% SDS-PAGE, blotted and probed with anti-GFP monoclonal antibody JL-8 (Clontech, Palo Alto, Calif.) and visualized using a secondary antibody, goat anti-mouse-HRP (Sigma). Chemiluminescent signals were generated by incubation with the ECL reagent and the gels were exposed to X-ray film. Cell lines and culture

Several cell lines used herein in the examples originated in the inventors' laboratory or were obtained from other sources: mesenchymal MBA-13, MBA-15, 14F1.1, NIH/3T3, AC-6, AC-11 and FBMD-1 cells; control C2C12, 1C8, MPC-11 and AB-8 cells; and MC/9 mastocytoma cells.

The cell lines were cultured by standard procedures such as in DMEM containing 10% FCS or with RPMI 1640 (GIBCO) containing 7% FCS, 2 mM L-glutamine, 5×10⁻⁵ M 2-mercaptoethanol and 1 mM sodium pyruvate. Other cell lines were cultured in DMEM containing 10% FCS and D-9 medium containing IL-3 and IL-4, or cultured in DMEM containing 20% FCS.

Primary Cell Culture

(i) Bone marrow: Mouse bone marrow cells were obtained from femur and tibia of 1-2 week old female C57BL/6 mice. Bone marrow cells were removed aseptically by flushing culture medium through the marrow cavity using a 1 ml syringe fitted with a 27-gauge needle. 1×10⁷ cells/ml were seeded in DMEM with 20% FCS (Bio Lab, Israel) and cultured for 4-5 days at 37° C. and 5% CO₂ atmosphere. The plates were washed and covered with fresh culture medium. After 3 weeks, a monolayer was formed. The cells were passaged monthly at a split ratio of 1:10 using 0.5% trypsin (Sigma, St. Louis, Mo.) containing 0.02% EDTA.

(ii) Fetal fibroblast: Mouse embryos were cut into small pieces in PBS solution and treated with 0.5% trypsin and 0.02% EDTA at 37° C. for 15 minutes. The supernatant was collected and treated again with trypsin for 30 minutes. The cell suspension obtained was then washed a few times, resuspended in DMEM containing 10% FCS to a final concentration of 10⁶ cells/ml, and cultured for 4-5 days at 37° C. and 5% CO₂ atmosphere. When a fibroblast monolayer was formed, it was trypsinized for 5 minutes, and the cells were washed and resuspended as indicated before. This cell suspension (2×10⁵ cells/ml) was cultured again for 4-5 days and then collected.

(iii) Thymus and liver cells were obtained from Balb/c mice, 6-10 weeks old.

Proliferation Assay

Stromal cells were seeded at 1×10⁵ cells/ml on a 96-well round-bottom microplate (Falcon, Calif.) for 48 hours at 37° C. in a humidified atmosphere of 10% CO₂ in air. The subconfluent cultures were supplemented with the relevant antibodies and incubated for an additional 48 hours. The cells were then pulsed with 1 μCi/well of [³H]-thymidine (Nuclear Research Center, Negev, Israel). After 24 hours, the cells were harvested, and the incorporation of tritiated thymidine was determined. Briefly, the supernatants were aspirated, the cell monolayer was washed repeatedly with PBS to remove excess thymidine and extracted with 0.1N NaOH 0.2 ml/well. A volume of 0.1 ml of the cell extract was added to 3 ml scintillation liquid/vial (Quicksafe, A. Zinsser, Germany) and the radioactivity was counted in a liquid scintillation analyzer (1600TR, Packard, Conn.). [³H]-thymidine incorporation reflecting the DNA synthesis was expressed as the stimulation index and was calculated as the ratio of the mean cpm of the experimental samples to the mean cpm of the control sample. Untreated cells or cells treated with irrelevant antibody served as control.

Antibodies

The following monoclonal antibodies (mAbs) were used in the experiments: fluorescein isothiocyanate (FITC)-mAb anti-CD3ε (clone 145-2C11); low azide no endotoxin or FITC-conjugated hamster anti-mouse TCRβ (clone H57-597); phycoerythrin (PE)-conjugated hamster anti-mouse TCRγδ (clone GL-3). All antibodies were purchased from PharMingen, San Diego, Calif. Goat anti-human IgM (Kalestab, Denmark), FITC-conjugated goat anti-mouse (Sigma, Israel) and mouse anti-rat IgG (Jackson Immunoresearch Labs, West Grove, Pa.) served as control antibodies. Hybridoma supernatants of anti-TCRβ (clone H57-597) and anti-CD3ε (clone 145-2C11) were used for activity assays. FITC-conjugated goat anti-hamster IgG was purchased from Jackson Immunoresearch Labs. Anti-rabbit FITC Fab fragments was used as a second antibody to detect staining with rabbit polyclonal anti-peptide 1121 and anti-Jβ2.6 [SEQ ID NO: 37] peptide antibodies.

Flow Cytometry

Cells were washed with PBS without Ca⁺² and Mg⁺² containing 0.02% sodium azide and incubated for 30 minutes at 4° C. with FITC-conjugated anti-mouse CD3ε (clone 145-2C11) or FITC-conjugated TCRβ (clone H57-597) or anti-Jβ2.6 [SEQ ID NO: 37] peptide antibodies. As second antibody for anti-Jβ2.6 [SEQ ID NO: 37] peptide antibody, FITC-conjugated donkey anti-rabbit IgG was used (Jackson Immunoresearch Labs). For intracellular staining, cells were fixed and stained with TCRβ using the Cytoperm kit (Serotec, UK). In all experiments, cells stained with isotype-matched control immunoglobulins were also prepared as negative controls for the surface and the intracellular staining. After washing with PBS, cells were analyzed for fluorescence with a FACScan (Becton Dickinson) with logarithmic intensity scales. In most cases, 5×10³ cells were scored using Lysis II software (Becton Dickinson).

Immunofluorescence

Stromal cells were seeded at 10⁵ cells/ml in chamber slides (Labtec slides: Nunc, USA) (400 μl/well) and incubated for 24 hours at 37° C. in a humidified atmosphere of 10% CO₂ in air. The slides were washed in PBS (without Mg⁺² and Ca⁺²) and were either unfixed or fixed in 3.7% paraformaldehyde in PBS for 20 minutes and permeabilized with 0.5% Triton X-100 in fixing solution for 2 minutes. The cells were washed with PBS for 5 minutes and blocked with normal sheep serum for 45 minutes and then stained with the relevant antibodies for 30 minutes. After incubation, the cells were washed with PBS, stained with the fluorescent second antibody for 30 minutes, washed, embedded in 50% glycerol in PBS and cover slips were mounted and sealed. Fluorescence was examined using a Zeiss fluorescence microscope (Zeiss, Oberkochen, Germany).

RNA Isolation and Northern Blotting

Total RNA was extracted by Tri-Reagent (Molecular Research Center, Cincinnati, Ohio). For Northern blotting, poly A+mRNA was obtained using oligo dT magnetic columns (Promega, Madison, Wis.). 5-30 μg mRNA was Northern blotted and probed using standard techniques with probes for the following regions: TCR Cα, TCR Cα and CD3ε. The probes were labeled with [³²P]-dCTP by random priming (Prime-a-Gene, Promega, Madison, Wis.), prehybridized at 42° C. in 50% deionized formamide, 2×Denhardt's solution, 0.1% SDS, 5×SSPE, 100 mg/ml boiled salmon sperm DNA. Hybridization was performed at the same conditions with 1×10⁶ cpm/ml labeled probe. Filters were washed twice with 1×SSC, 0.1% SDS at 42° C. for 30 minutes and then washed twice with 0.1×SSC, 0.1% SDS at 55° C. for 30 minutes.

PCR Analysis

Total RNAs were reverse transcribed to cDNAs by incubating purified total RNA at 37° C. for 60 minutes in the presence of MMLV reverse transcriptase. The primer pairs used for CD3ε were as follows: sense primer, 5′-TGCCCTCTAGACAGTGACG-3′; and antisense primer 5′-CTTCCGGTTCCGGTTCGGA-3′. The TCR derived primer pairs used were as follows:

| | |
|---|---|
| Cβ5: | 1′-ATGTGACTCCACCCAAGGTCTCCTTGTTTG-3′; |
| Cβ5: | 2′-AAGGCTACCCTCGTGTGCTTGGCCAGGGGC-3′; |
| Cβ5: | 3′-CATCCTATCATCAGGGGGTTCTGTCTGCAA-3′; |
| Cβ5: | 5′-CATCCTATCATCAGGGGGTTCTGTCTGCAA-3′; |
| Cβ5: | 6′-TTCAGAGTCAAGGTGTCAACGAGGAAGG-3′; |
| Cα1: | 5′-AAGATCCTCGGTCTCAGGACAGCACC-3′; |
| Cα2: | 5′-ACTGTGCTGGACATGAAAGCTATGGATTCC-3′; |
| | or |
| Tm: | 5′-GATTTAACCTGCTCATGACG-3′. |

For PCR, thirty cycles of amplification were carried out using the following conditions for each cycle: denaturation at 94° C. for 5 minutes, annealing at 58° C. for 2 minutes, and extension at 72° C. for 2 minutes.

Rapid Amplification of 5′ and 3′ Ends (RACE)

5′ and 3′ RACE was performed for the cloning of the TCR Cβ chain of MBA-13 cells using the Marathon cDNA amplification kit (Clontech, Palo Alto, Calif.). Adaptor ligated cDNA was prepared from MBA-13 mRNA according to manufacturers' directions. Hotstart-Touchdown PCR was performed as follows: 94° C. for 5 minutes (x1 cycle), 94° C. for 1 minute and 74° C. for 3 minutes (x5 cycles), 94° C. for 1 minute and 70° C. for 3 minutes (x15 cycles), 94° C. for 1 minute and 68° C. for 3 minutes (x10 cycles). Specific primers were used paired to the adaptor primer of the kit. The RACE products were cloned into the pGEM-T plasmid (Promega) and transfected into E. coli JM109 cells (Promega). DNA was purified and sequenced using an automated DNA sequencer (Applied Biosystems 373A, New England Nuclear, Boston, Mass.).

Statistics

Data are presented as the mean ± standard error of the mean. Student's t-test was performed to determine significance.

### Example 1

Jβ2.6 Nucleotide Sequence

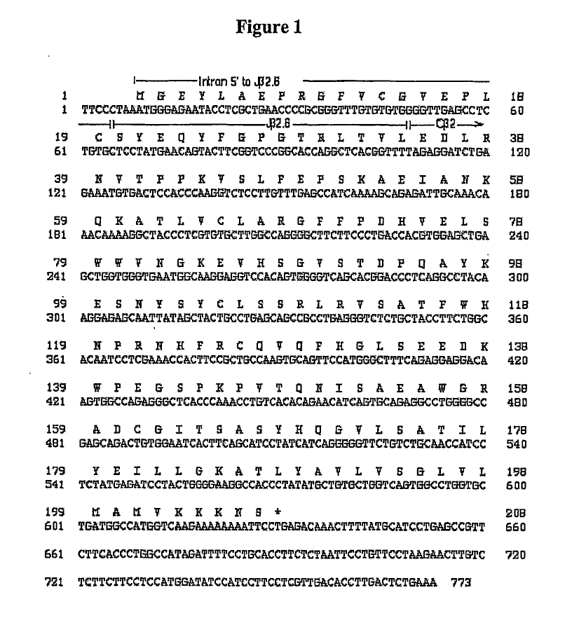

FIG. 1 shows the nucleotide sequence of a cDNA that was cloned from the stromal/mesenchymal cell line, MBA-13, that shows a Jβ2.6 flanked by an intronic J (J^{int}J2.6-C).

The J^{int}-JD2.6-C mRNA encodes a putative protein that according to available literature (Irving, 1998) should be capable of being expressed on the cell surface. We therefore raised polyclonal rabbit antibodies by immunizing with a synthetic peptide sequence based on the Jβ2.6 intronic peptidic sequence [SEQ ID NO:2] as follows LAEPRGFVCGVE [SEQ ID NO:37]. For immunization, peptide SEQ ID NO:37 was conjugated to KLH and was injected into 2 New Zealand rabbits using Complete Freund's Adjuvant for the first immunization and Incomplete Freund's Adjuvant for additional boosts. Pre-immune serum was collected before the first immunization and immune sera were collected after the additional boosts. Reactivity of the serum with the peptide SEQ ID NO:37 was tested by ELISA. The serum was purified on a peptide affinity column (eluted in 0.1M glycine pH 2.5 and dialyzed to PBS). The purified anti-peptide SEQ ID NO:37 antibody was also tested by ELISA.

### Example 2

Cytometric Analysis of J^{int}J-Cβ₂ Surface Protein Expression and mRNA Transcription

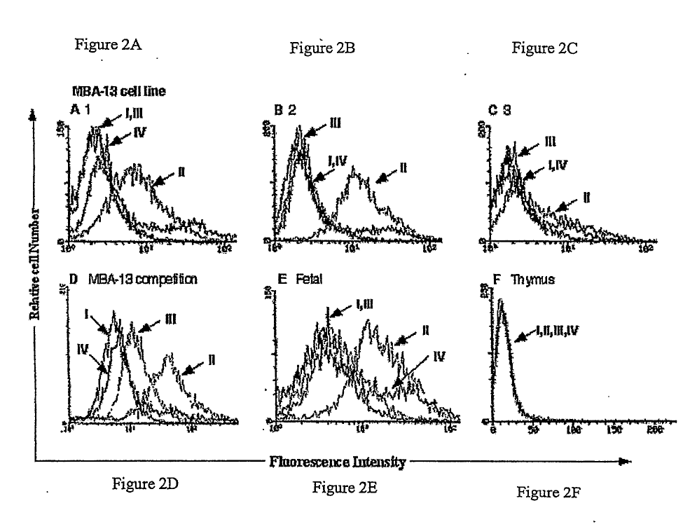

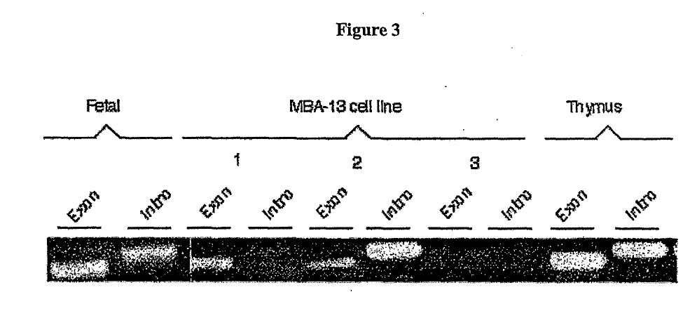

The immunized rabbit serum was processed by isolating the specific antibodies using a column of the immunizing peptide SEQ ID NO:37. These antibodies were then tested for their ability to recognize various cell types: MBA-13 cell strains 1, 2 and 3, mouse embryonic fibroblasts (MEF) and thymus cells as shown in FIG. 2. Whereas thymus cells were not stained (FIG. 2F), as judged by FACS analysis, two strains of the MBA-13 mesenchymal cell lines showed prominent cell surface staining by the polyclonal antibodies (FIGS. 2A, 2B). On the other hand, one clone of the MBA-13 cell line was negative (FIG. 2C). A striking finding is that we found correlation between the expression of the J^{int}-Jβ2.6-C mRNA (FIG. 3) and the reactivity of the antiserum with the stromal cells. Thus, the two cell strains that expressed J^{int}-Jβ2.6-C mRNA, also reacted with the antibody whereas one of the strains that did not show any J^{int}-Jβ2.6-C mRNA, also did not give any signal in flow cytometric analysis using the antibodies to the intronic peptide [SEQ ID NO:37] (FIG. 2C, clone 3).

The specificity of the detection of the antigen by the antiserum was further verified using competition assays with the soluble immunizing peptide that reduced the ability of the antiserum to stain the cells (FIG. 2D). This strongly supports the conclusion that J^{int}-Jβ2.6-C protein is present on the surface of the MBA-13 cells. It is noteworthy that thymocytes do express J^{int}-Jβ2.6-C on the mRNA level but are not reactive with the antibody (FIGS. 2F and 3). This in fact should be expected since most thymocytes express productively rearranged TCRβ and suppression of the expression of other transcripts should occur. On the other hand, in mesenchymal cells that lack recombinases, no complete TCRβ molecules are formed, which allows the expression of the J^{int}-Jβ2.6-C protein.

The above findings were made using a permanent cell line (MBA-13) derived in our laboratory. We further aimed to find out whether primary mesenchymal cells also express the J^{int}-Jβ2.6-C mRNA. As shown in FIG. 2E and FIG. 3, primary fibroblasts from mouse embryo clearly express the gene both on the protein and mRNA levels.

### Example 3

Murine and Human Truncated TCRαβ Sequences

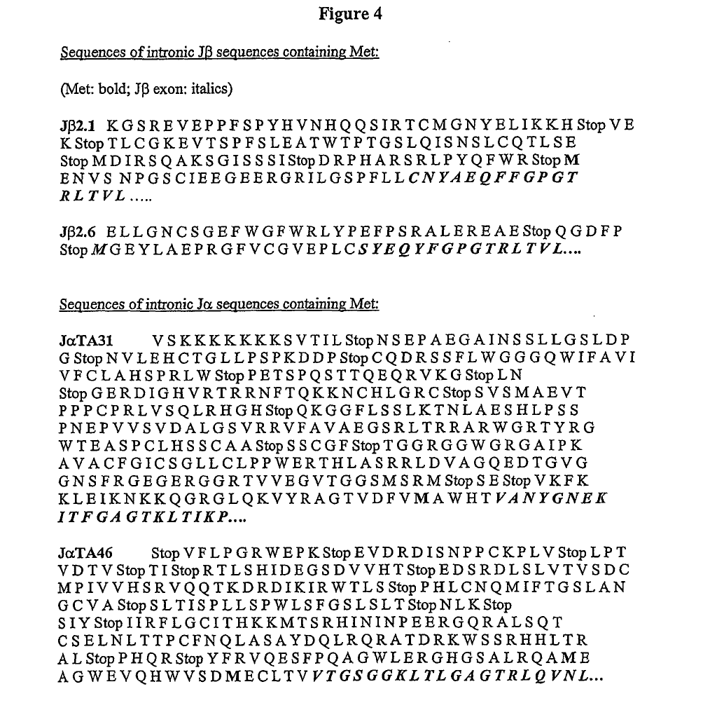

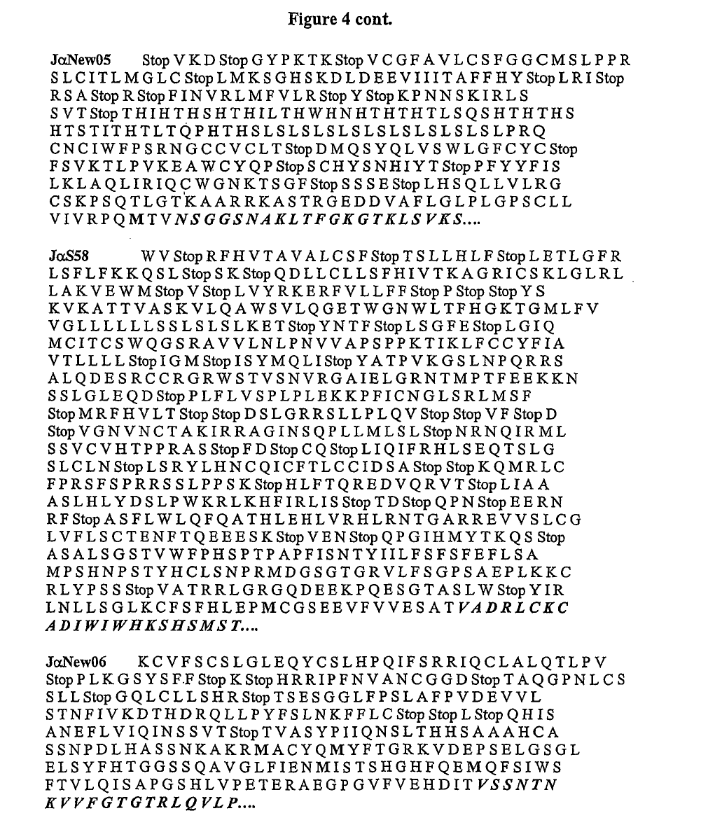

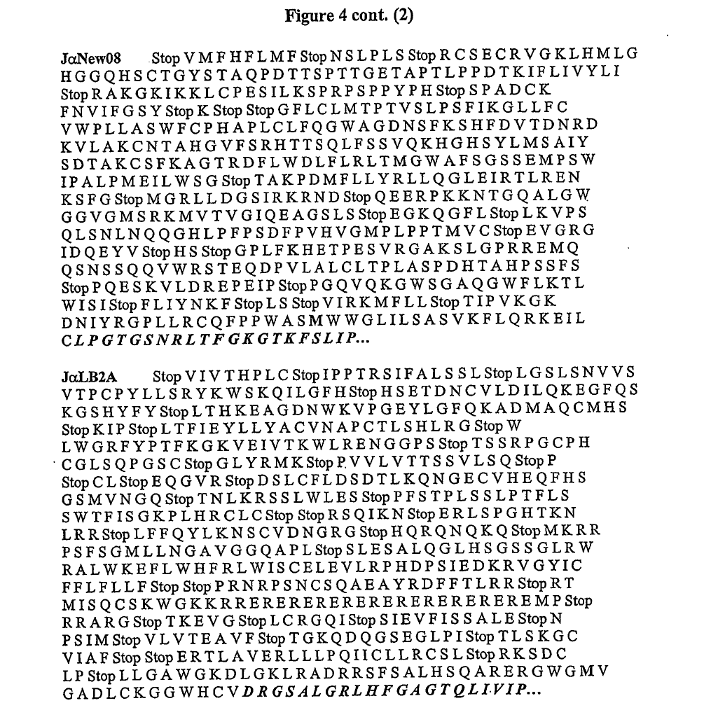

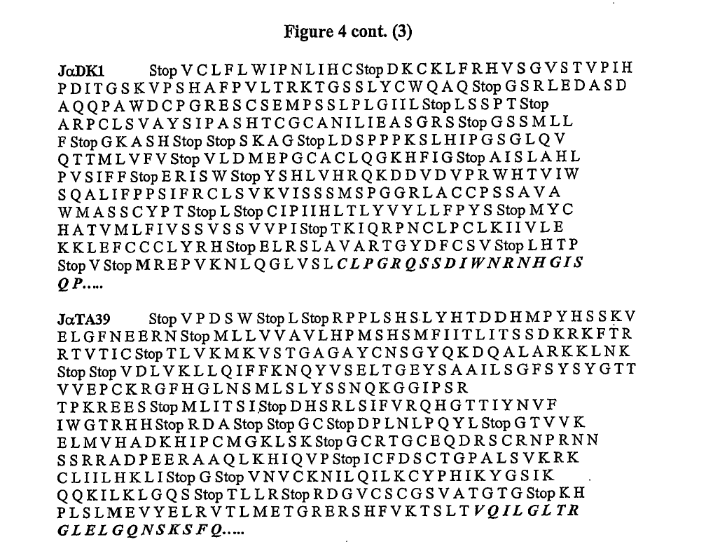

A database survey indicated that among the seven Jβs known, also Jβ2.1 can theoretically encode a molecule such as J^{int}-Jβ2.6-C. Indeed, PCR analysis using appropriate primers detected this mRNA in the MBA-13 cell line. Among the 47 possible Jαs, 9 could theoretically have a composition of intronic J with an in-frame methionine codon. These sequences are shown in FIG. 4 and include: JαTA31, JαTA46, JαNew05, JαS58, JαNew06, JαNew08, JαLB2A, JαDK1 and JαTA39. Preliminary PCR analysis indicates that at least some of these versions of the a chain also exist. In addition there are 3 possible Jα molecules initiated by a methionine from within the exonic coding region (data not shown).

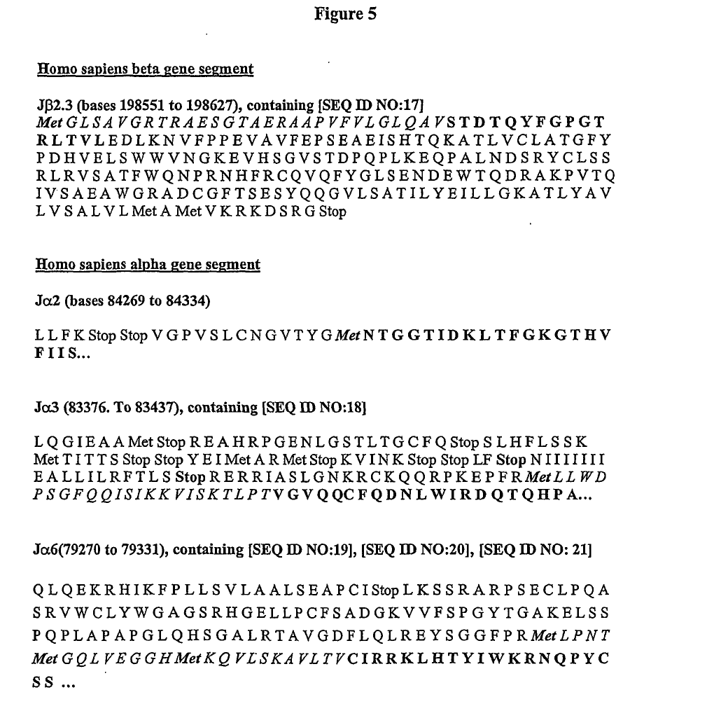

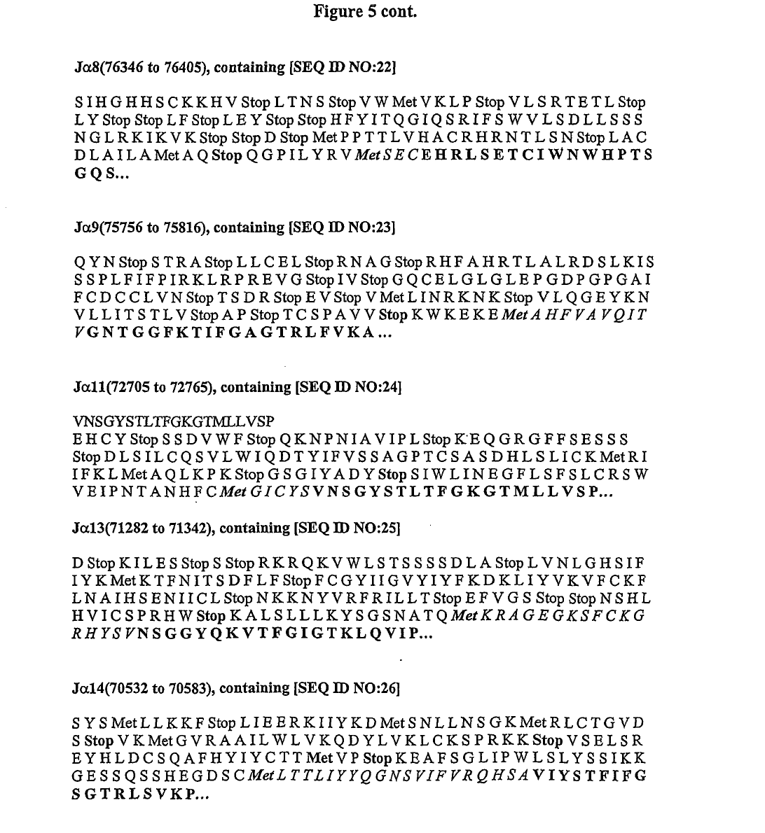

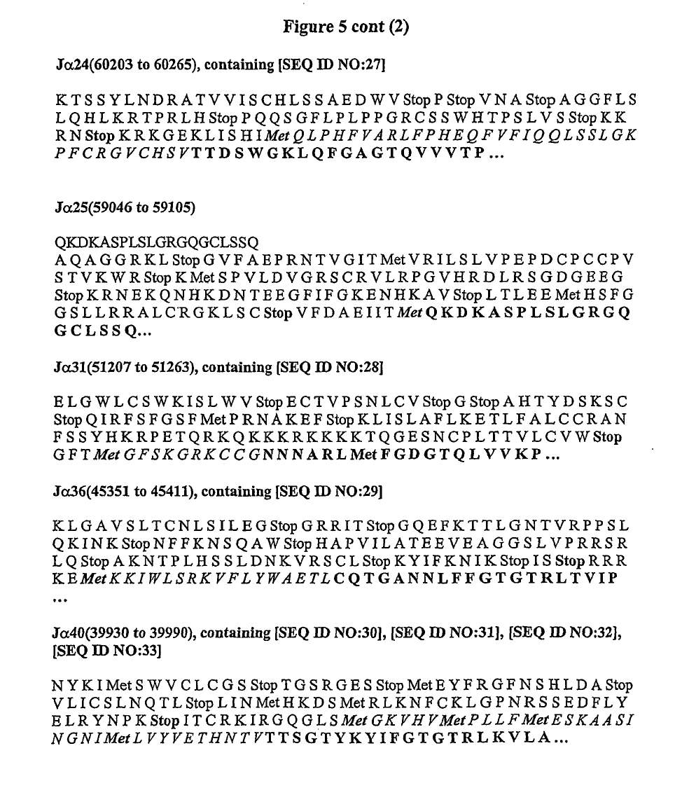

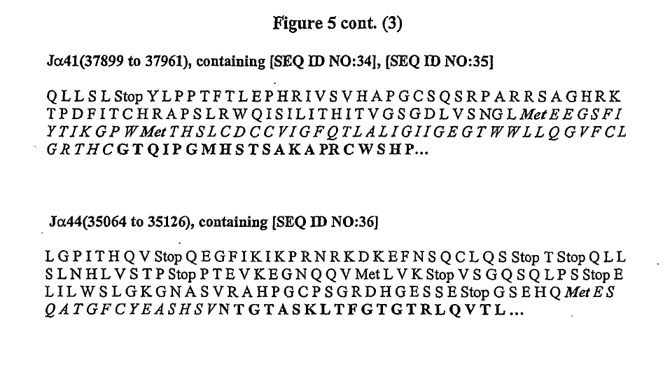

The following are the human sequences according to the present invention. In this example, the methionine initiating the open reading frame is shown in bold italics, the amino acids that are translated from an intronic sequence upstream to the J segments are shown in italics and the J segments are shown in bold, three dots denote the beginning of the C1 segments (FIG. 5).

### Example 4

### Subcloning of MBA-13 Cell Line

According to the present invention, the uncloned stromal/mesenchymal mouse MBA-13 cell line was subdivided into subclones that either express or do not express the molecules of interest, i.e. the J^{int}-Jβ2.6-C protein and mRNA, on the mRNA and antigenic protein levels. We therefore single cell cloned MBA-13 cells and obtained 8 different clonal populations by standard procedures. Out of these, 4 expressed the J^{int}-Jβ2.6-C protein (M-TCR⁺ clones C4, D10, B10, B1) and 4 were negative (M-TCR⁻ clones E4, C6, G1, B7).

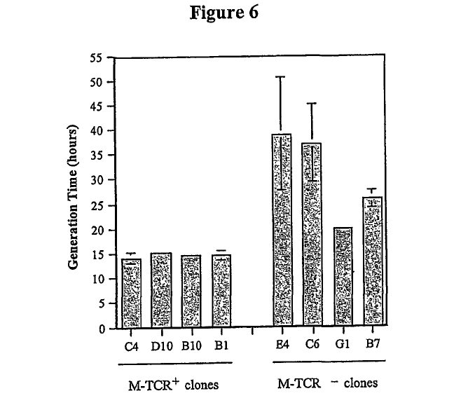

FIG. 6 shows that all the cells positive for J^{int}-Jβ2.6-C had a population generation time (doubling time) of 15 hrs or less, which is considered very fast for mesenchymal cells. On the other hand, although the negative clones showed variable results, all grew much slower and 2 clones had a very slow growth rate with doubling time between 36-38 hrs. It is therefore implied that the expression of the gene of interest correlates with fast growth rate and that lack of expression results in retarded growth. These results are supported by preliminary data indicating that antibodies to TCRβ constant region interfere with the growth of mesenchymal cells.

### Example 5

RT-PCR Analysis of TCR Expression

It is known in the art from T cell research that TCRβ can operate as a functional receptor and can cause apoptosis in the cells in which it is expressed. However, when pTα is coexpressed with TCRβ, pTα augments the function of TCRβ. In order to check if this was the case in our system, we examined the expression of the pTα in the mesenchymal cells. Indeed pTα is expressed by the MBA-13 cell line as judged by RT-PCR Thus, these mesenchymal cells seem to express a pTα/J^{int}-Jβ2.6-C complex which is structurally related to a reported TCR complex containing pTα and an experimentally truncated TCR (Irving, 1998). The latter complex has been shown to be sufficient for intracellular signaling suggesting that the complex in MBA-13 is likely to be effective in signal transduction.

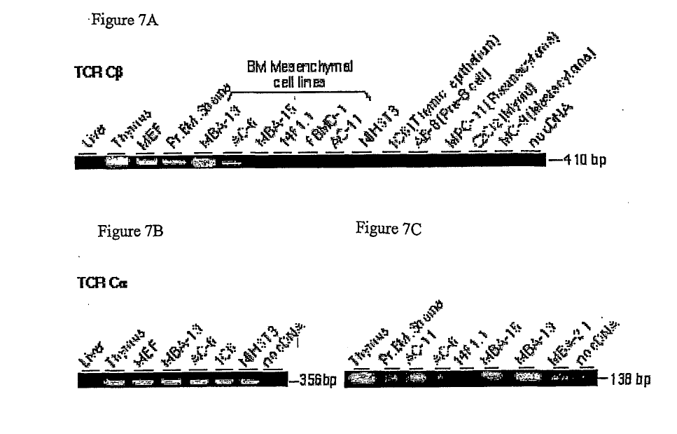

The study of expression of TCR was extended to a variety of stromal cell lines derived by the laboratory of the present inventors or obtained from other laboratories, as well as to primary stromal cells from the bone marrow and primary mesenchymal cells from mouse embryos. Specific stromal cell clones, but not all clones tested, expressed TCRβ. Similarly, TCRα was consistently found in particular stromal cell clones (e.g., the MBA-13 stromal cell line expressed both Cβ and Cα, whereas the MBA-15 stromal cell line did not express Cβ but was positive for Cα (FIGS. 7A-7C). Similar TCR amplified PCR products were observed in cultured primary embryo fibroblasts (FIGS. 7A-7C), indicating that the expression of TCR was not a bizarre characteristic of in vitro passaged stromal cell lines. Rather, TCR gene expression, as judged by PCR amplification, was common to primary mesenchymal and in vitro passaged cells of this origin. Indeed, bone marrow mesenchymal cells, seeded in vitro and selected by passaging to remove contaminating hemopoietic cells, also showed clear TCRαβ fragments of the expected sizes in PCR analysis. TCR gene expression was not found in B cells, mast cells or liver cells (FIGS. 7A-7B).

### Example 6

mRNA Expression of TCRCβ, TCRCα, and CD3ε

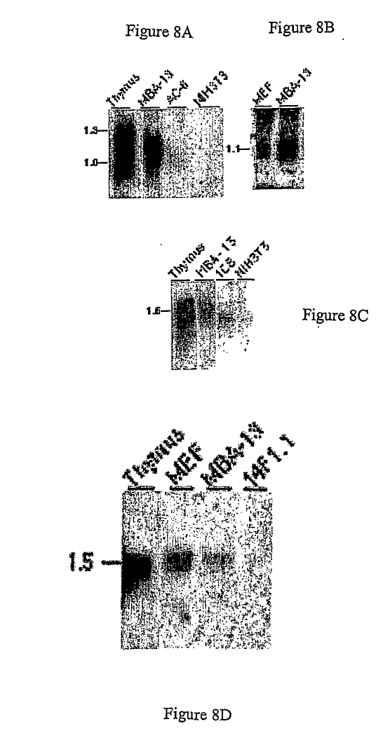

As shown in FIGS. 8A-8B, TCRαβ mRNA was detected in the MBA-13 stromal cell line and also in primary fetal and bone marrow fibroblast cultures. The sizes of the TCRα transcript corresponded to that found in thymic T cells, whereas the size of the mRNA detected by the TCRβ probe was about 1.1 kb as compared to 1.0 kb and 1.3 kb detected in the thymus. Of significance is that this shorter mRNA version was consistently found in different stromal cell lines, as well as in primary mesenchymal cells. A 1.0 kb mRNA species has been reported in bone marrow-derived immature precursor T cells. The relationship between the mesenchymal 1.1 kb mRNA species and that found in early bone marrow thymocytes remains to be examined.

The above data thus demonstrate that cells of mesenchymal origin do express TCR receptor complex on the mRNA level. In addition to expression of TCRαβ mRNA, expression of CD3ε, which is an essential component of the functional TCR complex, was observed (FIG. 8D). Both the size of the PCR amplified product and the mRNA detected by Northern blotting deviated slightly from the sizes detected in control T cell-derived cDNA.

### Example 7

Cytometric Analysis of CD3ε, TCRαβ and TCRγδ Antigen Expression by MBA-13 Cells

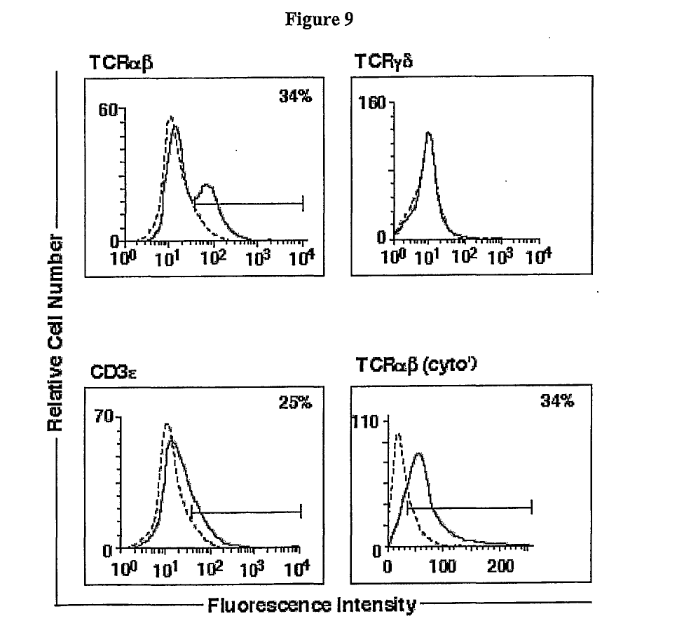

Flow cytometric analysis of stromal cells using an antibody to TCRαβ constant region indicated that 34% of the MBA-13 cell population was stained at low intensity fluorescence (FIG. 9). These stromal cells were negative when probed with antibodies to TCRγδ. Importantly, no TCRαβ was observed in cell lines that did not show TCRαβ mRNA. These data substantiate the above described results using antibodies to the intronic sequence of Jβ2.6.

### Example 8

Cytometric Analysis of a Mesenchymal Cell Surface Antigen Reactive with an Anti-TCRβ Antibody

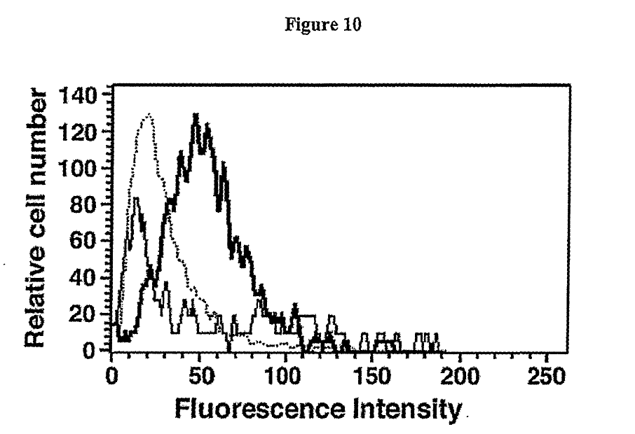

Furthermore, sequencing data of the PCR products from the TCRβ transcribed in mesenchymal cells confirmed that the TCRβ contains the entire C region as found in T cells. To test whether mesenchymal cells express a TCR protein, we used the H57-597 monoclonal antibody that identifies the C region. Flow cytometric analysis of MEF using this antibody demonstrated that MEF cells from wild type mice are clearly positive and express this antigen on the cell surface (FIG. 10). By contrast, no similar antigen was observed in MEF from TCRβ^{√-} mutant mice, that do not express TCRβ mRNA, providing genetic support for the existence of this TCR protein in mesenchymal cells.

### Example 9

Human TCR GFP-TCR Jβ2.3-Cβ

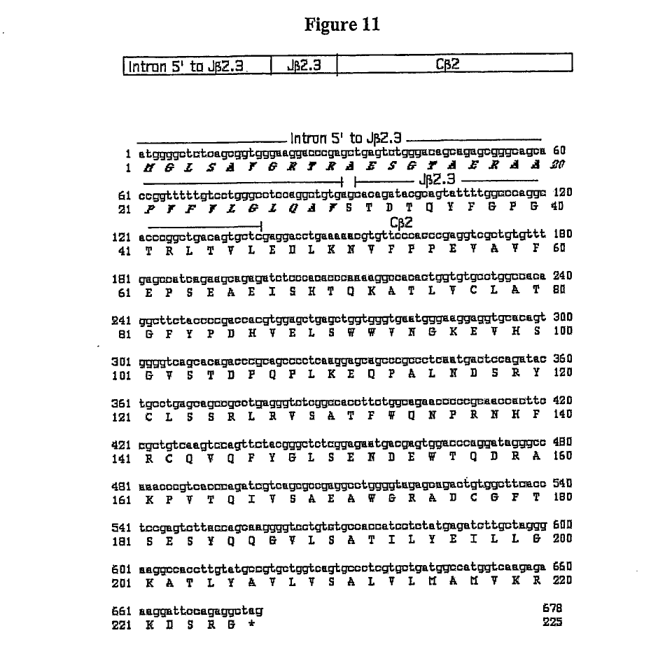

More support from a human system is gained from the cloning of the human TCR Jβ2.3-Cβ transcript from cDNA of cord blood mononuclear cells and amniotic fluid cells (FIG. 11). The cloned transcripts were sequenced and were found to be identical. The lines above the sequence indicate the boundaries of each segment. The predicted protein product is shown below the sequence. Bold font indicates an A to G transition that was found in both clones.

### Example 10

Expression of GFP-TCR Jβ2.3-Cβ and Recombinant Mesenchymal TCRβ (GFP-J^{int}-Jβ2.6-C)

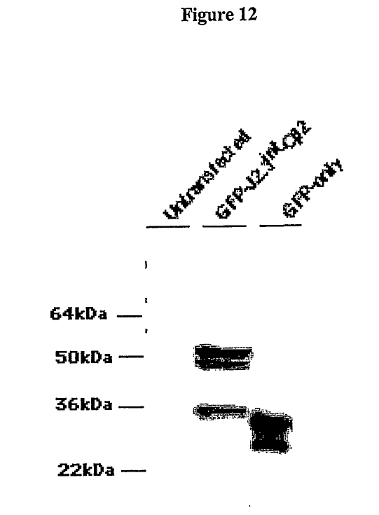

As an extension of the results obtained above, the expression of the fusion protein, GFP-TCR Jβ2.3-Cβ, in 293T transfected cells was determined by Western blot analysis. Each lane was loaded with lysate of 5×10⁵ cells. The GFP-TCR Jβ2.3-Cβ was detected with anti-GFP monoclonal antibody JL-8 (FIG. 12).

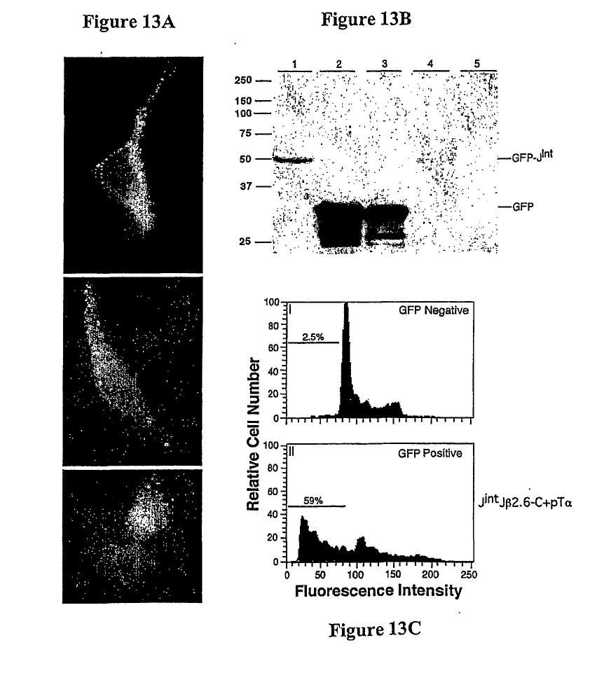

Next, we examined the results of overexpression of J^{int}-Jβ2.6-Cβ. A cDNA construct encoding a fusion protein of J^{int}-Jβ2.6-C N-terminally linked to green fluorescence protein (GFP) was transfected along with pTα into human 293T cells (FIG. 13). In this manner, overexpression of recombinant J^{int}-Jβ2.6-Cβ occurred in delicate dots which appeared to be cell membrane associated (FIG. 13A). Transfection with GFP-J^{int}-Jβ2.6-Cβ alone was insufficient in producing a similar dotted expression pattern and resulted in poor expression of the protein (compare lane 1 and 4, FIG. 13B) (however, further experiments done under different conditions have shown that it is possible to obtain overexpression of J^{int}-Jβ2.6-Cβ when transfected on its own). Similar results were obtained with the MBA-13 mesenchymal cell line, albeit with far lower transfection efficiency (not shown). These results are consistent with the fact that cell surface localization of TCRβ is dependent upon complex formation with pTα in preT cells. Flow cytometric analysis of the cells co-transfected with GFP-J^{int}-Jβ2.6-Cβ and pTα showed a dramatic shift of 59% of the population to sub-G1, indicating massive apoptosis (FIG. 3C-II). This indicates that the relative amount of this protein and the correct timing of its expression in the cell signal cell fate. Although these experiments show that pTα and J^{int}-Jβ2.6-Cβ form a minimal functional receptor complex, further investigations are required to determine the other possible components of the mesenchymal preT cell-like receptor.

### Example 11

Tumor Formation of MBA-13 Subclones

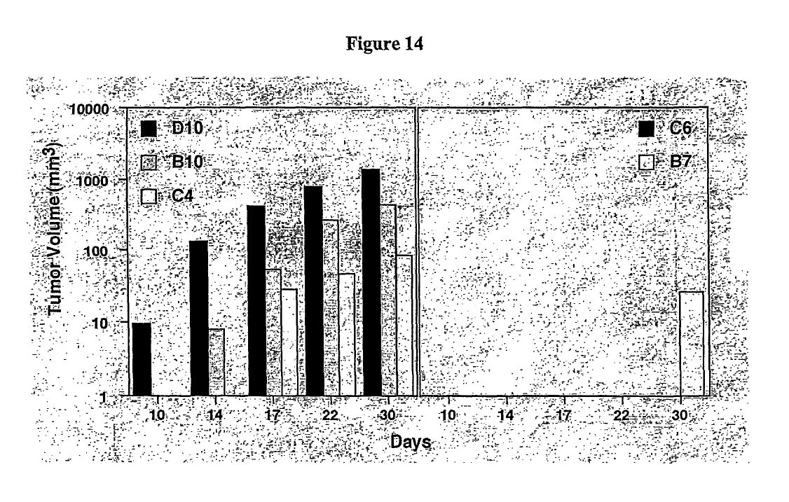

Finally, we examined the possible relevance of TCR expression by mesenchymal cells to their biological functions. As mentioned above, the MBA-13 cell line was single cloned by limiting dilutions and each clone was examined for expression of TCRβ mRNA. It was observed that the highly expressing clones (D10, B10 and C4) were also tumorigenic in vivo. (FIG. 14). Intradermal injection of these stromal cell clones into nude CD1 recipient mice resulted in tumor formation within a few weeks, only in the case of the fast growing clones (D10, B10 and C4). The slow growing clones (C6 and B7) injected into mice formed tumors at a low incidence and at one month following inoculation.

### Example 12

In Vivo Utility

The pharmaceutical compositions of the present invention can be used for treatment of diseases involving modulation of mesenchymal growth. By treatment of disease is meant prevention or amelioration of the disease or of symptoms associated with the disease, or minimizing subsequent worsening of the disease or of symptoms associated with the disease. The diseases and conditions to be treated include conditions in which it is preferable to inhibit mesenchymal growth including: cancer, especially in the case of metastasis to any organ, especially the bone marrow, nonmalignant proliferative diseases of any organ, especially the bone marrow, bone marrow defects resulting in hematological disorders such as anemias or leukemias and autoimmune diseases involving any organ, especially the bone marrow.

In addition, the present invention can be used for treatment of conditions where it is desirable to augment mesenchymal growth including autologous or allogeneic bone marrow transplantation, wound healing and autologous or allogeneic organ transplantation.

It will be appreciated that the most appropriate administration of the pharmaceutical compositions of the present invention will depend on the type of injury, disease or condition being treated. Thus, the treatment of an acute event will necessitate systemic administration of the active composition comparatively rapidly after induction of the injury. On the other hand, diminution of chronic degenerative damage will necessitate a sustained dosage regimen.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

Reference to known method steps, conventional method steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art

### REFERENCES

Abbas et al (Eds.) (1994). Cellular and Molecular Immunology Chapter II, W. B. Saunders Co. (Philadelphia, USA).

Bordignon C, Notarangelo L D, Nobili N, Ferrari G, Casorati G, Panina P, Mazzolari E, Maggioni D, Rossi C, Servida P, et al. (1995). Gene therapy in peripheral blood lymphocytes and bone marrow for ADA- immunodeficient patients. Science 270(5235):470-5

Calman A F, Peterlin B M (1986) Expression of T cell receptor genes in human B cells. J Exp Med 164(6):1940-57

Essand M, Vasmatzis G, Brinkmann U, Duray P, Lee B, Pastan I (1999).High expression of a specific T-cell receptor gamma transcript in epithelial cells of the prostate. Proc Natl Acad Sci USA 96(16):9287-92

Fagioli M, Care A, Ciccone E, Moretta L, Moretta A, Meccia E, Testa U, Falini B, Grignani F, Peschle C, et al. (1991). Molecular heterogeneity of the 1.0-kb T beta transcript in natural killer and gamma/delta lymphocytes. Eur. J Immunol. 21(6):1529-34

Irving B A, Alt F W, Killeen N (1998). Thymocyte development in the absence of pre-T cell receptor extracellular immunoglobulin domains. Science 280(5365):905-8

Jameson S C, Bevan M J (1995). T cell receptor antagonists and partial agonists. Immunity 2(1): 1-11

Kimoto, Y (1998). Expression of heavy-chain constant region of immunoglobulin and T-cell receptor gene transcripts in human non-hematopoietic tumor cell lines. Genes, Chromosomes, Cancer 22(1): 83-86.

Madrenas J, Pazderka F, Baergen C, Halloran P F (1991). Isolation of a murine renal cell population which expresses a truncated T-cell receptor-alpha mRNA. Transplant Proc 23(1 Pt 1):837-8

Madrenas J, Pazderka F, Parfrey N A, Halloran P F (1992). Thymus-independent expression of a truncated T cell receptor-alpha mRNA in murine kidney. J Immunol. 148(2):612-9

Madrenas J, Vincent D H, Kriangkum J, Elliott J F, Halloran P F (1994). Alternatively spliced, germline J alpha 11-2-C alpha mRNAs are the predominant T cell receptor alpha transcripts inmouse kidney. Mol Immunol. 31(13):993-1004

Qian L, Vu M N, Carter M S, Doskow J, Wilkinson M F (1993). T cell receptor-beta mRNA splicing during thymic maturation in vivo and in an inducible T cell clone in vitro. J Immunol 15;151(12):6801-14

Strominger J L (1989). Developmental biology of T cell receptors. Science; 244(4907):943-50

Wientroub S, Zipori D. (1996). “Stem Cell Culture” in: Principles of Bone Biology. J. Bilezikian, L. Raisz, G. Rodan, J. Markovac (eds). Academic Press, San Diego, pp 1267

Wolfgang C D, Essand M, Vincent J J, Lee B, Pastan I (2000). TARP: a nuclear protein expressed in prostate and breast cancer cells derived from an alternate reading frame of the T cell receptor gamma chain locus. Proc Natl Acad Sci USA 97(17):9437-42

Yoshikai Y, Anatoniou D, Clark S P, Yanagi Y, Sangster R, Van den Elsen P, Terhorst C, Mak T W (1984). Sequence and expression of transcripts of the human T-cell receptor beta-chain genes. Nature 312(5994):521-4

Zipori D (1989) Cultured stromal cell lines from hemopoietic tissues. In:Tavassoli M, ed., Blood Cell Formation: The Role of the Hemopoietic Microenvironment, Humana Press (Clifton, N.Y.), p. 287

Zipori D (1990). Stromal cells in tumor growth and regression. Cancer J 3: 164

Zipori D, Tamir M (1989). Stromal cells of hemopoietic origin. Int J Cell Cloning 7(5):281-91

1. An isolated polynucleotide comprising a transcript of a T cell receptor (TCR) gene, the polynucleotide lacking V region sequences and comprising a constant (C) domain and joining (J) region sequences, and a 5′ intronic J sequences upstream of the J region sequence including an in-frame methionine codon.

2. The polynucleotide according to claim 1, wherein the gene is a TCRβ gene.

3. The polynucleotide according to claim 2, wherein the joining (J) gene sequence is selected from Jβ2.1 and Jβ2.6.

4. The polynucleotide according to claim 3, wherein the joining (J) gene sequence is Jβ2.1 and said 5′ intronic J sequence including an in-frame methionine codon codes for a peptide of the sequence M EN V S N P G S C I E E G E E R G R I L G S P F L [SEQ ID NO:1].

5. The polynucleotide according to claim 3, wherein the joining (J) gene sequence is Jβ2.6 and said 5′ intronic J sequence including a methionine codon codes for a peptide of the sequence M G E Y L A E P R G F V C G V E P L C [SEQ ID NO:2].

6. The polynucleotide according to claim 1, comprising a 5′ intronic J sequence encoding a peptide selected from any one of SEQ ID Nos: 1-37.

7. The polynucleotide of claim 2 wherein the joining J gene sequence is the intronic Jβ2.3 gene sequence coding for the peptide:

| | |
|---|---|
| MGLSAVGRTRAESGTAERAAPVFVLGLQAV. | [SEQ ID NO:17] |

8. The polynucleotide according to claim 1, wherein the gene is a TCRα gene.

9. The cDNA molecule according to claim 8, wherein the joining (J) gene sequence is selected from human or murine Jα genes.

10. The cDNA molecule according to claim 9, wherein said 5′ intronic J sequence including an in-frame methionine codon is selected from the group consisting of:
(i) the intronic JαTA31 gene sequence coding for the peptide:

| | |
|---|---|
| MAWH; | [SEQ IN NO:3] |

(ii) the intronic JαTA46 gene sequence coding for the peptide:

| | |
|---|---|
| MEAGWEVQHWVSDMECLTV; | [SEQ IN NO:4] |

(iii) the intronic JαTA46 gene sequence coding for the peptide:

| | |
|---|---|
| MECLTV; | [SEQ IN NO:5] |

(iv) the intronic JαNew05 gene sequence coding for the peptide:

| | |
|---|---|
| MTV; | [SEQ IN NO:6] |

(v) the intronic JαS58 gene sequence coding for the peptide:

| | |
|---|---|
| MCGSEEVFVVESA; | [SEQ IN NO:7] |

(vi) the intronic JαNew06 gene sequence coding for the peptide:

| | |
|---|---|
| MACYQMYFTGRKVDEPSELGSGLELSYFHTGGSSQAV | [SEQ IN NO:8] |
| GLFIENMISTSHGHFQEMQFSIWSFTVLQISAPGSHL | |
| VPETERAEGPGVFVEHDI; | |

(vii) the intronic JαNew06 gene sequence coding for the peptide:

| | |
|---|---|
| MYFTGRKVDEPSELGSGLELSYFHTGGSSQAVGLFIE | [SEQ IN NO:9] |
| NMISTSHGHFQEMQFSIWSFTVLQISAPGSHLVPETE | |
| RAEGPGVFVEHDI; | |

(viii) the intronic JαNew06 gene sequence coding for the peptide:

| | |
|---|---|
| MISTSHGHFQEMQFSIWSFTVLQISAPGSHLVPETE | [SEQ IN NO:10] |
| RAEGPGVFVEHDI; | |

(xi) the intronic JαNew06 gene sequence coding for the peptide:

| | |
|---|---|
| MQFSIWSFTVLQISAPGSHLVPETERAEGPGVFVEH | [SEQ IN NO:11] |
| DI; | |

(x) the intronic JαNew08 gene sequence coding for the peptide:

| | |
|---|---|
| MWWGLILSASVKFLQRKEILC; | [SEQ IN NO:12] |

(xi) the intronic JαLB2A gene sequence coding for the peptide:

| | |
|---|---|
| MVGADLCKGGWHCV; | [SEQ IN NO:13] |

(xii) the intronic JαDK1 gene sequence coding for the peptide:

| | |
|---|---|
| M R E P V K N L Q G L V S; | [SEQ IN NO:14] |

(xiii) the intronic JαTA39 gene sequence coding for the peptide:

| | |
|---|---|
| M E V Y E L R V T L M E T G R E R S | [SEQ IN NO:15] |
| H F V K T S L; | |
| and | |

(xvi) the intronic JαTA39 gene sequence coding for the peptide:

| | |
|---|---|
| M E T G R E R S H F V K T S L. | [SEQ IN NO:16] |

11. The polynucleotide according to claim 8, wherein 5′ intronic J sequence including an in-frame methionine codon is selected from the group consisting of:
(i) the intronic Jα3 gene sequence coding for the peptide:

| | |
|---|---|
| MLLWDPSGFQQISIKKVISKTLPT; | [SEQ IN NO: 18] |

(ii) the intronic Jα6 gene sequence coding for the peptide:

| | |
|---|---|
| MLPNTMGQLVEGGHMKQVLSKAVLTV; | [SEQ IN NO:19] |

(iii) the intronic Jα6 gene sequence coding for the peptide:

| | |
|---|---|
| MGQLVEGGHMKQVLSKAVLTV; | [SEQ IN NO:20] |

(iv) the intronic Jα6 gene sequence coding for the peptide:

| | |
|---|---|
| MKQVLSKAVLTV; | [SEQ IN NO:21] |

(v) the intronic Jα8 gene sequence coding for the peptide:

| | |
|---|---|
| MSEC; | [SEQ IN NO:22] |

(vi) the intronic Jα9 gene sequence coding for the peptide:

| | |
|---|---|
| MAHFVAVQITV; | [SEQ IN NO:23] |

(vii) the intronic Jα11 gene sequence coding for the peptide:

| | |
|---|---|
| MGICYS; | [SEQ IN NO:24] |

(viii) the intronic Jα13 gene sequence coding for the peptide:

| | |
|---|---|
| MKRAGEGKSFCKGRHYSV; | [SEQ IN NO:25] |

(ix) the intronic Jα14 gene sequence coding for the peptide:

| | |
|---|---|
| MLTTLIYYQGNSVIFVRQHSA; | [SEQ IN NO:26] |

(x) the intronic Jα24 gene sequence coding for the peptide:

| | |
|---|---|
| MQLPHFVARLFPHEQFVFIQQLSSLGKPFCRGVCH | [SEQ IN NO:27] |
| SV; | |

(xi) the intronic Jα31 gene sequence coding for the peptide:

| | |
|---|---|
| M G F S K G R K C C G; | [SEQ IN NO:28] |

(xii) the intronic Jα36 gene sequence coding for the peptide:

| | |
|---|---|
| M K K I W L S R K V F L Y W A E T L; | [SEQ IN NO:29] |

(xiii) the intronic Jα40 gene sequence coding for the peptide:

| | |
|---|---|
| M G K V H V M P L L F M E S K A A S | [SEQ IN NO:30] |
| I N G N I M L V Y V E T H N T V; | |

(xiv) the intronic Jα40 gene sequence coding for the peptide:

| | |
|---|---|
| M P L L F M E S K A A S I N G N I M | [SEQ IN NO:31] |
| L V Y V E T H N T V; | |

(xv) the intronic Jα40 gene sequence coding for the peptide:

| | |
|---|---|
| M E S K A A S I N G N I M L V Y V E | [SEQ IN NO:32] |
| T H N T V; | |

(xvi) the intronic Jα40 gene sequence coding for the peptide:

| | |
|---|---|
| MLVYVETHNTV; | [SEQ IN NO:33] |

(xvii) the intronic Jα41 gene sequence coding for the peptide:

| | |
|---|---|
| MEEGSFIYTIKGPWMTHSLCDCCVIGFQTLALIGII | [SEQ IN NO:34] |
| GEGTWWLLQGVFCLGRTHC; | |

(xviii) the intronic Jα41 gene sequence coding for the peptide:

| | |
|---|---|
| MTHSLCDCCVIGFQTLALIGIIGEGTWWLLQGVFCL | [SEQ IN NO:35] |
| GRTHC | |
| and | |

(xix) the intronic Jα44 gene sequence coding for the peptide:

| | |
|---|---|
| MESQATGFCYEASHSV. | [SEQ IN NO:36] |

12. An antisense polynucleotide of the polynucleotides according to claim 1.

13. An expression vector comprising a polynucleotide according to claim 1.

14. A host cell comprising a vector according to claim 13, wherein the host is a mammalian cell.

15. Transfected mesenchymal human cells according to claim 14.

16. A polypeptide encoded by a polynucleotide according to claims 1.

17. A polynucleotide comprising SEQ ID NO:38 or SEQ ID NO:39.

18. A synthetic peptide deduced from an intronic J sequence of a TCR.

19. The synthetic peptide according to claim 18 selected from the group consisting of any one of SEQ ID Nos:1-16 or SEQ ID Nos. 17-36.

20. An antibody raised against a peptide according to claim 18.

21. An antibody raised against a peptide according to claim 19.

22. A method for inducing mesenchymal cell growth comprising administering to a subject in need thereof transfected mesenchymal human cells comprising a polynucleotide according to claim 1, in an amount effective to induce mesenchymal cell growth.

23. The method according to claim 22, wherein the method induces wound healing.

24. A method for suppressing mesenchymal cell growth comprising administering to a subject in need thereof transfected mesenchymal human cells comprising a DNA molecule according to claim 12, in an amount effective to suppress mesenchymal cell growth.

25. The method according to claim 24, wherein the method suppresses carcinomas.

26. A method of marking mesenchymal cells comprising applying an antibody according to claim 20 to mesenchymal cells in an amount effective to mark the cells.
